(19) United States
(12) Patent Application Publication
(10) Pub. No.: US 2026/0043011 A1
(43) Pub. Date: Feb. 12, 2026

(54) Luminescent Protein

(71) Applicant: OSAKA UNIVERSITY, Osaka (JP)

(72) Inventors: Takeharu Nagai, Osaka (JP); Soki Tanaka, Osaka (JP); Kenji Osabe, Osaka (JP); Tetsuyuki Entani, Osaka (JP)

(73) Assignee: OSAKA UNIVERSITY, Osaka (JP)

(21) Appl. No.: 19/102,204

(22) PCT Filed: Aug. 8, 2023
(86) PCT No.: PCT/JP2023/028990

§ 371 Date: Feb. 7, 2025

(30) Foreign Application Priority Data
Aug. 10, 2022 (JP) 2022-128319

(51) Int. Cl. C12N 9/02; C12N 15/70

(52) U.S. Cl. CPC C12N 9/0069; C12N 15/70; C12N 2800/101

(57) ABSTRACT

The present disclosure, in one aspect, provides a modified luciferase with improved luminescence intensity, and use thereof. The present disclosure relates to a modified luciferase derived from a luminescent mushroom or a functional fragment thereof, including a mutation introduced into arginine or lysine corresponding to position 26 and/or a mutation introduced into valine corresponding to position 161 of amino acid sequence shown in SEQ ID NO: 1, and having enhanced luminescence compared with a luciferase before the mutation is introduced. The present disclosure also relates to an isolated nucleic acid encoding the modified luciferase or functional fragment thereof of the present disclosure.

### SEQUENCE LISTING SUBMISSION VIA PATENT CENTER

The contents of the electronic sequence listing (sequencelisting.xml; Size: 99,295 bytes; and Date of Creation: Aug. 7, 2023) is herein incorporated by reference in its entirety.

### TECHNICAL FIELD

The present disclosure relates to a modified luciferase and use thereof.

### BACKGROUND ART

Molecular imaging is a technique that observes the movements and changes of various molecules in vivo within a living subject. Chemiluminescence imaging is one such molecular imaging technique. Chemiluminescence imaging uses a luminescence phenomenon (luciferin-luciferase reaction) caused by an oxidation reaction of a luminescent substrate (luciferin) catalyzed by a chemiluminescent protein (luciferase) to perform in vivo observation.

Many organisms, such as fireflies, Vargula hilgendorfii, and luminescent mushrooms, have the ability to chemically emit light. Such chemiluminescence reactions are caused by a chemiluminescent protein (luciferase), which is one of the luminescent proteins, and its luminescent substrate (luciferin). The structures of these proteins and substrates vary widely from species to species. Currently, various luminescent proteins are applied as reporters and probes in life science research.

In recent years, it has been discovered that the luminescent substrate for luciferase in luminescent mushrooms (luminescent fungi) is 3-hydroxyhispidin, and furthermore, luciferase genes of luminescent mushrooms have been identified (Non-Patent Documents 1 to 3 and Patent Document 1).

### PRIOR ART DOCUMENTS

### Non-Patent Documents

Non-Patent Document 1: Kotlobay et al., Genetically encodable bioluminescent system from fungi, 12728-12732, PNAS, Dec. 11, 2018, vol. 115, no. 50, www.pnas.org/cgi/doi/10.1073/pnas.1803615115
Non-Patent Document 2: Khakhar et al., Building customizable auto-luminescent luciferase-based reporters in plants, eLife 2020; 9:e52786., https://doi.org/10.7554/eLife.52786
Non-Patent Document 3: Mitiouchkina T, et al., Plants with genetically encoded autoluminescence. Nat Biotechnol. 2020 August; 38(8): 944-946 Patent Document
Patent Document 1: JP 2020-505028T

### DISCLOSURE OF INVENTION

### Problem to be Solved by the Invention

When luciferase is expressed in cells or the like and used as a reporter or a probe, it is necessary to add a luminescent substrate (luciferin) to the cells or the like. An approach to solving the problem of adding luciferin is autonomous luminescence imaging (autoluminescence imaging), in which a group of genes that constitute the biosynthetic cycle of luciferin is simultaneously introduced into a living organism, allowing for imaging to be performed. One such autoluminescence system is a system derived from a luminescent mushroom (fungal bioluminescence pathway: FBP). The FBP can be said to be particularly suitable for autoluminescence imaging in plants.

However, the luminescence intensity of autoluminescence systems derived from luminescent mushrooms imposes limitations on the observation of physiological phenomena occurring within or between cells, and therefore, there is a need to improve the luminescence intensity of autoluminescence systems derived from luminescent mushrooms.

The present disclosure, in one aspect, provides a modified luciferase with improved luminescence intensity, and use thereof.

### Means for Solving Problem

In one aspect, the present disclosure relates to a modified luciferase derived from a luminescent mushroom or a functional fragment thereof, including a mutation introduced into at least one of arginine or lysine corresponding to position 26 and valine corresponding to position 161 in an amino acid sequence shown in SEQ ID NO: 1, and having enhanced luminescence compared with a luciferase before the mutation is introduced or the wild-type luciferase derived from the luminescent mushroom.

In one aspect, the present disclosure relates to an isolated nucleic acid encoding the modified luciferase or functional fragment thereof of the present disclosure.

In one aspect, the present disclosure relates to an isolated nucleic acid encoding a modified luciferase or a functional fragment thereof having any amino acid sequence selected from the group consisting of (a) to (c) below, and a mutation introduced into arginine or lysine corresponding to position 26 of the amino acid sequence shown in SEQ ID NO: 1 and/or a mutation introduced into valine corresponding to position 161 of the amino acid sequence shown in SEQ ID NO: 1, in the amino acid sequence selected from the group consisting of (a) to (c) below:
(a) an amino acid sequence of a luciferase derived from a luminescent mushroom, which is set forth in SEQ ID NO: 1, 3, 5, 7, 9, 11, 13, 15, or 17;
(b) an amino acid sequence that is at least 80% identical to the amino acid sequence set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine; and
(c) an amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 of the amino acid sequence shown in SEQ ID NO: 1 is arginine or lysine, and an amino acid corresponding to position 161 of the amino acid sequence shown in SEQ ID NO: 1 is valine.

In one aspect, the present disclosure relates to a modified luciferase or a functional fragment thereof having any amino acid sequence selected from the group consisting of (a) to (c) below, and a mutation introduced into arginine or lysine corresponding to position 26 of the amino acid sequence shown in SEQ ID NO: 1 and/or a mutation introduced into valine corresponding to position 161 of the amino acid sequence shown in SEQ ID NO: 1, in the amino acid sequence selected from the group consisting of (a) to (c) below:
(a) an amino acid sequence of a luciferase derived from a luminescent mushroom, which is set forth in SEQ ID NO: 1, 3, 5, 7, 9, 11, 13, 15, or 17;
(b) an amino acid sequence that is at least 80% identical to the amino acid sequence set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine; and
(c) an amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine.

### Effects of the Invention

According to the present disclosure, in one aspect, it is possible to provide a modified luciferase with improved luminescence intensity.

### BRIEF DESCRIPTION OF DRAWINGS

FIG. 1 shows a comparison of luminescence intensities of the wild type and variants of luciferase from Mycena chlorophos in Escherichia coli colonies in Example 1.

FIG. 2 shows an alignment of sequences of luciferases (nine types) from luminescent mushrooms in Example 2.

FIG. 3 shows a comparison of luminescence intensities of the wild type and variants of luciferase from Neonothopanus nambi in E. coli colonies in Example 3.

### DESCRIPTION OF THE INVENTION

The present disclosure is based on the finding that a modified luciferase obtained by inserting a mutation into specific amino acids (an amino acid corresponding to position 26 in the amino acid sequence shown in SEQ ID NO: 1 and an amino acid corresponding to position 161 in the amino acid sequence shown in SEQ ID NO: 1) of a luciferase derived from a luminescent mushroom has improved luminescence intensity compared with the luciferase before the mutation is introduced.

The term “luciferase” as used in the present disclosure refers to an enzyme that catalyzes an oxidation reaction of a luminescent substrate (luciferin). The energy generated in the oxidation process of luciferin catalyzed by luciferase is emitted as light. The term “modified luciferase of the present disclosure” refers to a luciferase that is modified according to the present disclosure, specifically, a luciferase in which a mutation is introduced into at least one of arginine or lysine corresponding to position 26 and/or valine corresponding to position 161 in the amino acid sequence shown in SEQ ID NO: 1.

The term “luminescent mushroom” as used in the present disclosure refers to a mushroom that produces luminescence, specifically, a mushroom that emits green light through an enzymatic reaction catalyzed by luciferase. In one or more embodiments, examples of the luminescent mushroom include Mycena chlorophos, Neonothopanus nambi, Armillaria gallica, Armillarni mellea, Armillarni ostoyae, Omphalotus olearius, Panellus stipticus, Mycena citricolor; and Neonothopanus gardneri.

The term “luciferase derived from a luminescent mushroom” as used in the present disclosure refers to a luciferase extracted from a luminescent mushroom. In one or more embodiments, examples of the luciferase derived from a luminescent mushroom include luciferase from Mycena chlorophos, luciferase from Neonothopanus nambi, luciferase from Armillaria gallica, luciferase from Armillaria mellea, luciferase from Armillaria ostoyae, luciferase from Mycena citricolor; luciferase from Neonothopanus gardner; luciferase from Omphalotus olearius, and luciferase from Panellus stipticus.

In the present disclosure, the terms “polynucleotide” and “nucleic acid” are deemed synonymous. In one or more embodiments, the polynucleotide may be any type of nucleotide sequence. In one or more embodiments, examples of the polynucleotide include a synthetic RNA sequence, a synthetic DNA sequence, a cDNA sequence, a partial genomic DNA sequence, and the like.

In the present disclosure, the term “polypeptide” is synonymous with the term “protein”. The term “polypeptide” refers to a chain of continuous residues where the α-amino group and the carboxyl group of adjacent amino acids are linked by peptide bonds. In one or more embodiments, the polypeptide may include structures such as a sugar chain and an isoprenoid.

The term “isolate” or “isolated” as used in the present disclosure may encompass that the nucleic acid or polypeptide of the present disclosure has been prepared or isolated in vitro or substantially purified from an in-vitro substance. Also, the term “isolate” or “isolated” means that the nucleic acid or polypeptide of the present disclosure is substantially separate from other substances and components that would accompany them in their natural state, or is in an environment different from the environment in which they are present in their natural state.

The term “isolated polypeptide” as used in the present disclosure may encompass a polypeptide, peptide, or protein encoded by cDNA or recombinant RNA, as well as combinations thereof.

In the present disclosure, “isolated nucleic acid of the present disclosure” may be simply referred to as “nucleic acid of the present disclosure”.

The term “functional fragment” as used in the present disclosure refers to a portion or region of a luciferase capable of catalyzing an oxidation reaction of luciferin that produces luminescence, with the portion or region having the same or similar activity as that of the full-length luciferase. In one or more embodiments, the functional fragment of the present disclosure may have at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, at least 99%, or 100% of the activity of the full-length luciferase.

The term “nucleic acid (nucleotide sequence) encoding a polypeptide” as used in the present disclosure means that a polypeptide encoded by the nucleic acid (nucleotide sequence) is produced from the nucleotide sequence through transcription and translation of mRNA. In one or more embodiments, the nucleotide sequence encoding a polypeptide may include a sequence containing an intron.

The term “amino acid corresponding to an amino acid at position 26 and/or position 161 in the amino acid sequence shown in SEQ ID NO: 1” as used in the present disclosure refers to an amino acid identified by homology analysis as corresponding to an amino acid at position 26 and/or position 161 in the amino acid sequence shown in SEQ ID NO: 1. In one or more embodiments, homology analysis can be performed using a publicly available sequence comparison program. In one or more embodiments, examples of methods for homology analysis include pairwise sequence alignment, multiple sequence alignment (such as the ClustalW method), and other similar methods. Based on these methods, specifically by performing, for example, multiple alignment, a person skilled in the art can determine the “amino acid corresponding to position 26 and/or position 161 in SEQ ID NO: 1” in an analysis target amino acid sequence by using the amino acid sequence shown in SEQ ID NO: 1 as a reference sequence.

Note that in multiple alignment, gaps may be present in one or more embodiments.

As used in the present disclosure, the term “mutation” of an amino acid sequence refers to substitution, deletion, or insertion of an amino acid residue. In one or more embodiments, the mutation is a substitution or a deletion, preferably a substitution.

The term “expression cassette” as used in the present disclosure refers to an expression unit that includes an expression control region (regulatory element) functional in a host cell and a polynucleotide operably linked to the expression control region. The phrase “operably linked” as used in the present disclosure means that the functional expression control region (regulatory element) in a host cell and the polynucleotide are physically or functionally linked to each other. In one or more embodiments, the expression cassette includes the expression control region (regulatory element) and the nucleic acid (polynucleotide) of the present disclosure that are linked to each other using a genetic engineering method.

In one or more embodiments, the expression cassette of the present disclosure may be in the form of an expression vector. An expression vector is usually introduced into a cell as an extrachromosomal element and may thus have an origin of replication to ensure its replication in the host cell. In one or more embodiments, the nucleic acid (polynucleotide) of the present disclosure is functionally linked to an expression regulatory sequence, in the expression vector. In one or more embodiments, examples of the expression regulatory sequence include a promoter, an enhancer, a transcription terminator, an operator, a repressor, a silencer, a silencing suppressor, an insulator, an inducer, a start codon, an intron splicing signal, a stop codon, and the like.

The term “enhanced luminescence” as used in the present disclosure means that, in one aspect, the luminescence intensity has been increased compared with that before the introduction of a mutation. In one or more embodiments, it means that the luminescence intensity of the luciferase of the present disclosure is at least 1%, at least 2%, at least 3%, at least 5%, at least 10%, at least 20%, at least 25%, at least 50%, at least 75%, at least 90%, at least 100%, at least 200%, at least 300%, or at least 400% higher than the luminescence intensity of the luciferase before the mutation is introduced (reference luciferase). The term “luciferase before the mutation is introduced (reference luciferase)” as used in the present disclosure refers to a luciferase that has not been modified (subjected to amino acid sequence mutation) according to the present disclosure, or in other words, a luciferase that retains its amino acid sequence before modification according to the present disclosure.

### Modified Luciferase

In one aspect, the present disclosure relates to a modified luciferase or a functional fragment thereof. In one or more embodiments, the modified luciferase of the present disclosure achieves the effect of having enhanced luminescence compared with a luciferase before modification according to the present disclosure (introduction of a mutation according to the present disclosure).

In one or more embodiments, the modified luciferase of the present disclosure includes a mutation introduced into arginine or lysine corresponding to position 26 of the amino acid sequence shown in SEQ ID NO: 1 and/or a mutation introduced into valine corresponding to position 161 of the amino acid sequence shown in SEQ ID NO: 1, and has enhanced luminescence compared with the luciferase before the mutation is introduced.

In one or more embodiments, the modified luciferase of the present disclosure is a variant obtained by modifying a luciferase from a luminescent mushroom that has the amino acid sequence as set forth in SEQ ID NO: 19 or 29. In one or more embodiments, the modified luciferase of the present disclosure has the amino acid sequence as set forth in SEQ ID NO: 19 or 29, with a mutation introduced into arginine or lysine at position 5 and/or a mutation introduced into valine at position 140 in the amino acid sequence as set forth in SEQ ID NO: 19 or 29. Note that, in one or more non-limiting embodiments, at least one or both of X at position 29 and X at position 130 in SEQ ID NOs: 19 and 29 may be gaps (amino acid deletions).

In one or more embodiments, the modified luciferase of the present disclosure has any amino acid sequence selected from the group consisting of (a) to (c) below, and a mutation introduced into arginine or lysine corresponding to position 26 of the amino acid sequence shown in SEQ ID NO: 1 and/or a mutation introduced into valine corresponding to position 161 of the amino acid sequence shown in SEQ ID NO: 1 in the amino acid sequence selected from the group consisting of (a) to (c) below:
(a) An amino acid sequence of a luciferase derived from a luminescent mushroom, which is set forth in SEQ ID NO: 1, 3, 5, 7, 9, 11, 13, 15, or 17.
(b) An amino acid sequence that is at least 80% identical to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine. In one or more embodiments, the sequence identity is at least 85%, at least 90%, at least 91%, at least 92%, at least 93%, at least 94%, at least 95%, at least 96%, at least 97%, at least 98%, or at least 99%.
(c) An amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine. In one or more embodiments, the number of amino acid substitutions, deletions, insertions, and/or additions is 1, 2, 3, 4, 5, 6, 7, 8, 9, 10, or 11.

In one or more embodiments, the modified luciferase of the present disclosure may include a homolog that may be subjected to a conservative substitution.

The amino acid sequence as set forth in SEQ ID NO: 1, 3, 5, 7, 9, 11, 13, 15, or 17 is the amino acid sequence of a luciferase from a luminescent mushroom. SEQ ID NO: 1 shows the amino acid sequence of luciferase from Mycena chlorophos, SEQ ID NO: 3 shows the amino acid sequence of luciferase from Neonothopanus nambi; SEQ ID NO: 5 shows the amino acid sequence of luciferase from Armillaria gallica, SEQ ID NO: 7 shows the amino acid sequence of luciferase from Armillaria mellea, SEQ ID NO: 9 shows the amino acid sequence of luciferase from Armillaria ostoyae, SEQ ID NO: 11 shows the amino acid sequence of luciferase from Mycena citricolor; SEQ ID NO: 13 shows the amino acid sequence of luciferase from Neonothopanus gardneri; SEQ ID NO: 15 shows the amino acid sequence of luciferase from Omphalotus olearius, and SEQ ID NO: 17 shows the amino acid sequence of luciferase from Panellus stipticus.

In one or more embodiments, the modified luciferase of the present disclosure is a luciferase having a mutation introduced into arginine or lysine corresponding to position 26 of the amino acid sequence shown in SEQ ID NO: 1 and/or a mutation introduced into valine corresponding to position 161 in the amino acid sequence shown in SEQ ID NO: 1 in luciferase from Mycena chlorophos(SEQ ID NO: 1), luciferase from Neonothopanus nambi (SEQ ID NO: 3), luciferase from Armillaria gallica (SEQ ID NO: 5), luciferase from Armillaria mellea (SEQ ID NO: 7), luciferase from Armillaria ostoyae (SEQ ID NO: 9), luciferase from Mycena citricolor (SEQ ID NO: 11), luciferase from Neonothopanus gardneri (SEQ ID NO: 13), luciferase from Omphalotus olearius (SEQ ID NO: 15), or luciferase from Panellus stipticus (SEQ ID NO: 17). In one or more embodiments, only the mutation at the amino acid (arginine or lysine) corresponding to position 26 of the amino acid sequence shown in SEQ ID NO: 1 may be introduced, only the mutation at the amino acid (valine) corresponding to position 161 of the amino acid sequence shown in SEQ ID NO: 1 may be introduced, or both of the mutations at the amino acid (arginine or lysine) corresponding to position 26 and the amino acid (valine) corresponding to position 161 of the amino acid sequence shown in SEQ ID NO: 1 may be introduced.

The amino acid corresponding to the amino acid (arginine) at position 26 of the amino acid sequence shown in SEQ ID NO: 1 is arginine at position 40 in luciferase from Neonothopanus nambi, arginine at position 42 in luciferase from Armillaria gallica, arginine at position 42 in luciferase from Armillaria mellea, arginine at position 42 in luciferase from Armillaria ostoyae, lysine at position 27 in luciferase from Mycena citricolor; arginine at position 36 in luciferase from Neonothopanus gardneri; arginine at position 29 in luciferase from Omphalotus olearius, or arginine at position 25 in luciferase from Panellus stipticus.

In the present disclosure, in one or more embodiments, an post-mutation amino acid to which the amino acid corresponding to the amino acid (arginine) at position 26 of the amino acid sequence shown in SEQ ID NO: 1 is mutated may be a naturally occurring amino acid other than arginine or lysine, or a non-naturally occurring amino acid. In one or more embodiments, the post-mutation amino acid may be an amino acid with an uncharged side chain. In one or more embodiments, the post-mutation amino acid may be an amino acid with an uncharged side chain having 3 or 4 carbon atoms. In one or more embodiments, the post-mutation amino acid may be leucine, isoleucine, glutamine, or the like.

In one or more embodiments, the modified luciferase of the present disclosure is an isolated polypeptide that has the amino acid sequence shown in SEQ ID NO: 1, and a mutation introduced into arginine at position 26, the mutation being R26L, R26I, or R26Q. In one or more embodiments, the modified luciferase of this aspect has enhanced luminescence compared with the wild-type luciferase (luciferase from Mycena chlorophos) having the amino acid sequence shown in SEQ ID NO: 1.

In one or more embodiments, the modified luciferase of the present disclosure is an isolated polypeptide that has the amino acid sequence shown in SEQ ID NO: 3, and a mutation introduced into arginine at position 40, which corresponds to position 26 of the amino acid sequence shown in SEQ ID NO: 1, the mutation being R40L, R40I, or R40Q. In one or more embodiments, the modified luciferase of this aspect has enhanced luminescence compared with the wild-type luciferase (luciferase from Neonothopanus nambi) having the amino acid sequence shown in SEQ ID NO: 3.

In one or more embodiments, the modified luciferase of the present disclosure is an isolated polypeptide that has the amino acid sequence shown in SEQ ID NO: 5, and a mutation introduced into arginine at position 42, which corresponds to position 26 of the amino acid sequence shown in SEQ ID NO: 1, the mutation being R42L, R42I, or R42Q. In one or more embodiments, the modified luciferase of this aspect has enhanced luminescence compared with the wild-type luciferase (luciferase from Armillaria gallica) having the amino acid sequence shown in SEQ ID NO: 5.

In one or more embodiments, the modified luciferase of the present disclosure is an isolated polypeptide that has the amino acid sequence shown in SEQ ID NO: 7, and a mutation introduced into arginine at position 42, which corresponds to position 26 in the amino acid sequence shown in SEQ ID NO: 1, the mutation being R42L, R42I, or R42Q. In one or more embodiments, the modified luciferase of this aspect has enhanced luminescence compared with the wild-type luciferase (luciferase from Armillaria mellea) having the amino acid sequence shown in SEQ ID NO: 7.

In one or more embodiments, the modified luciferase of the present disclosure is an isolated polypeptide that has the amino acid sequence shown in SEQ ID NO: 9, and a mutation introduced into arginine at position 42, which corresponds to position 26 of the amino acid sequence shown in SEQ ID NO: 1, the mutation being R42L, R42I, or R42Q. In one or more embodiments, the modified luciferase of this aspect has enhanced luminescence compared with the wild-type luciferase (luciferase from Armillaria ostoyae) having the amino acid sequence shown in SEQ ID NO: 9.

In one or more embodiments, the modified luciferase of the present disclosure is an isolated polypeptide that has the amino acid sequence shown in SEQ ID NO: 11, and a mutation introduced into lysine at position 27, which corresponds to position 26 of the amino acid sequence shown in SEQ ID NO: 1, the mutation being K27L, K27I, or K27Q. In one or more embodiments, the modified luciferase of this aspect has enhanced luminescence compared with the wild-type luciferase (luciferase from Mycena citricolor) having the amino acid sequence shown in SEQ ID NO: 11.

In one or more embodiments, the modified luciferase of the present disclosure is an isolated polypeptide that has the amino acid sequence shown in SEQ ID NO: 13, and a mutation introduced into arginine at position 36, which corresponds to position 26 in the amino acid sequence shown in SEQ ID NO: 1, the mutation being R36L, R36I, or R36Q. In one or more embodiments, the modified luciferase of this aspect has enhanced luminescence compared with the wild-type luciferase (luciferase from Neonothopanus gardneri) having the amino acid sequence shown in SEQ ID NO: 13.

In one or more embodiments, the modified luciferase of the present disclosure is an isolated polypeptide that has the amino acid sequence shown in SEQ ID NO: 15, and a mutation introduced into arginine at position 29, which corresponds to position 26 of the amino acid sequence shown in SEQ ID NO: 1, the mutation being R29L, R29I, or R29Q. In one or more embodiments, the modified luciferase of this aspect has enhanced luminescence compared with the wild-type luciferase (luciferase from Omphalotus olearius) having the amino acid sequence shown in SEQ ID NO: 15.

In one or more embodiments, the modified luciferase of the present disclosure is an isolated polypeptide that has the amino acid sequence shown in SEQ ID NO: 17, and a mutation introduced into arginine at position 25, which corresponds to position 26 of the amino acid sequence shown in SEQ ID NO: 1, the mutation being R25L, R25I, or R25Q. In one or more embodiments, the modified luciferase of this aspect has enhanced luminescence compared with the wild-type luciferase (luciferase from Panellus stipticus) having the amino acid sequence shown in SEQ ID NO: 17.

The amino acid corresponding to the amino acid (valine) at position 161 of the amino acid sequence shown in SEQ ID NO: 1 is valine at position 175 in luciferase from Neonothopanus nambi, valine at position 177 in luciferase from Armillaria gallica, valine at position 177 in luciferase from Armillaria mellea, valine at position 177 in luciferase from Armillaria ostoyae, valine at position 160 in luciferase from Mycena citricolor valine at position 171 in luciferase from Neonothopanus gardneri valine at position 164 in luciferase from Omphalotus olearius, or valine at position 160 in luciferase from Panellus stipticus.

In the present disclosure, in one or more embodiments, the post-mutation amino acid to which the amino acid corresponding to the amino acid (valine) at position 161 of the amino acid sequence shown in SEQ ID NO: 1 is mutated may be a naturally occurring amino acid other than valine, or a non-naturally occurring amino acid. In one or more embodiments, the post-mutation amino acid may be an amino acid with an uncharged side chain. In one or more embodiments, the post-mutation amino acid may be an amino acid with an uncharged side chain having 0 or 1 carbon atom. In one or more embodiments, the post-mutation amino acid may be alanine, serine, or glycine.

In one or more embodiments, the modified luciferase of the present disclosure is an isolated polypeptide that has the amino acid sequence shown in SEQ ID NO: 1, and a mutation introduced into valine at position 161, the mutation being V161A, V161S, or V161G. In one or more embodiments, the modified luciferase of this aspect has enhanced luminescence compared with the wild-type luciferase (luciferase from Mycena chlorophos) having the amino acid sequence shown in SEQ ID NO: 1.

In one or more embodiments, the modified luciferase of the present disclosure is an isolated polypeptide that has the amino acid sequence shown in SEQ ID NO: 3, and a mutation introduced into valine at position 175, which corresponds to position 161 of the amino acid sequence shown in SEQ ID NO: 1, the mutation being V175A, V175S, or V175G. In one or more embodiments, the modified luciferase of this aspect has enhanced luminescence compared with the wild-type luciferase (luciferase from Neonothopanus nambi) having the amino acid sequence shown in SEQ ID NO: 3.

In one or more embodiments, the modified luciferase of the present disclosure is an isolated polypeptide that has the amino acid sequence shown in SEQ ID NO: 5, and a mutation introduced into valine at position 177, which corresponds to position 161 of the amino acid sequence shown in SEQ ID NO: 1, the mutation being V177A, V177S, or V177G. In one or more embodiments, the modified luciferase of this aspect has enhanced luminescence compared with the wild-type luciferase (luciferase from Armillaria gallica) having the amino acid sequence shown in SEQ ID NO: 5.

In one or more embodiments, the modified luciferase of the present disclosure is an isolated polypeptide that has the amino acid sequence shown in SEQ ID NO: 7, and a mutation introduced into valine at position 177, which corresponds to position 161 of the amino acid sequence shown in SEQ ID NO: 1, the mutation being V177A, V177S, or V177G. In one or more embodiments, the modified luciferase of this aspect has enhanced luminescence compared with the wild-type luciferase (luciferase from Armillaria mellea) having the amino acid sequence shown in SEQ ID NO: 7.

In one or more embodiments, the modified luciferase of the present disclosure is an isolated polypeptide that has the amino acid sequence shown in SEQ ID NO: 9, and a mutation introduced into valine at position 177, which corresponds to position 161 of the amino acid sequence shown in SEQ ID NO: 1, the mutation being V177A, V177S, or V177G. In one or more embodiments, the modified luciferase of this aspect has enhanced luminescence compared with the wild-type luciferase (luciferase from Armillaria ostoyae) having the amino acid sequence shown in SEQ ID NO: 9.

In one or more embodiments, the modified luciferase of the present disclosure is an isolated polypeptide that has the amino acid sequence shown in SEQ ID NO: 11, and a mutation introduced into valine at position 160, which corresponds to position 161 in the amino acid sequence shown in SEQ ID NO: 1, the mutation being V160A, V160S, or V160G. In one or more embodiments, the modified luciferase of this aspect has enhanced luminescence compared with the wild-type luciferase (luciferase from Mycena citricolor) having the amino acid sequence shown in SEQ ID NO: 11.

In one or more embodiments, the modified luciferase of the present disclosure is an isolated polypeptide that has the amino acid sequence shown in SEQ ID NO: 13, and a mutation introduced into valine at position 171, which corresponds to position 161 of the amino acid sequence shown in SEQ ID NO: 1, the mutation being V171A, V171S, or V171G. In one or more embodiments, the modified luciferase of this aspect has enhanced luminescence compared with the wild-type luciferase (luciferase from Neonothopanus gardneri) having the amino acid sequence shown in SEQ ID NO: 13.

In one or more embodiments, the modified luciferase of the present disclosure is an isolated polypeptide that has the amino acid sequence shown in SEQ ID NO: 15, and a mutation introduced into valine at position 164, which corresponds to position 161 of the amino acid sequence shown in SEQ ID NO: 1, the mutation being V164A, V164S, or V164G. In one or more embodiments, the modified luciferase of this aspect has enhanced luminescence compared with the wild-type luciferase (luciferase from Omphalotus olearius) having the amino acid sequence shown in SEQ ID NO: 15.

In one or more embodiments, the modified luciferase of the present disclosure is an isolated polypeptide that has the amino acid sequence shown in SEQ ID NO: 17, and a mutation introduced into valine at position 160, which corresponds to position 161 of the amino acid sequence shown in SEQ ID NO: 1, the mutation being V160A, V160S, or V160G. In one or more embodiments, the modified luciferase of this aspect has enhanced luminescence compared with the wild-type luciferase (luciferase from Panellus stipticus) having the amino acid sequence shown in SEQ ID NO: 17.

In one or more embodiments, in order to obtain even higher luminescence intensity, it is preferable that the modified luciferase of the present disclosure includes mutations introduced into both the arginine or lysine corresponding to position 26 and the valine corresponding to position 161 of the amino acid sequence shown in SEQ ID NO: 1.

In one or more embodiments, the modified luciferase of the present disclosure is an isolated polypeptide that has the amino acid sequence shown in SEQ ID NO: 1, and mutations introduced into arginine at position 26 and valine at position 161 in the amino acid sequence, the mutations being R26L, R26I, or R26Q and V161A, V161S, or V161G. In one or more embodiments, the modified luciferase of this aspect has further enhanced luminescence compared with the wild-type luciferase (luciferase from Neonothopanus nambi) having the amino acid sequence shown in SEQ ID NO: 1.

In one or more embodiments, the modified luciferase of the present disclosure is an isolated polypeptide that has the amino acid sequence shown in SEQ ID NO: 3, and mutations introduced into arginine at position 40, which corresponds to position 26 of the amino acid sequence shown in SEQ ID NO: 1, and valine at position 175, which corresponds to position 161 of the amino acid sequence shown in SEQ ID NO: 1, the mutations being R40L, R40I, or R40Q and V175A, V175S, or V175G. In one or more embodiments, the modified luciferase of this aspect has further enhanced luminescence compared with the wild-type luciferase (luciferase from Armillaria gallica) having the amino acid sequence shown in SEQ ID NO: 3.

In one or more embodiments, the modified luciferase of the present disclosure is an isolated polypeptide that has the amino acid sequence shown in SEQ ID NO: 5, and mutations introduced into arginine at position 42, which corresponds to position 26 of the amino acid sequence shown in SEQ ID NO: 1, and valine at position 177, which corresponds to position 161 of the amino acid sequence shown in SEQ ID NO: 1, the mutations being R42L, R42I, or R42Q and V177A, V177S, or V177G. In one or more embodiments, the modified luciferase of this aspect has further enhanced luminescence compared with the wild-type luciferase (luciferase from Armillaria mellea) having the amino acid sequence shown in SEQ ID NO: 5.

In one or more embodiments, the modified luciferase of the present disclosure is an isolated polypeptide that has the amino acid sequence shown in SEQ ID NO: 7, and mutations introduced into arginine at position 42, which corresponds to position 26 off the amino acid sequence shown in SEQ ID NO: 1, and valine at position 177, which corresponds to position 161 of the amino acid sequence shown in SEQ ID NO: 1, the mutations being R42L, R42I, or R42Q and V177A, V177S, or V177G. In one or more embodiments, the modified luciferase of this aspect has further enhanced luminescence compared with the wild-type luciferase (luciferase from Armillaria ostoyae) having the amino acid sequence shown in SEQ ID NO: 7.

In one or more embodiments, the modified luciferase of the present disclosure is an isolated polypeptide that has the amino acid sequence shown in SEQ ID NO: 9, and mutations introduced into arginine at position 42, which corresponds to position 26 of the amino acid sequence shown in SEQ ID NO: 1, and valine at position 177, which corresponds to position 161 of the amino acid sequence shown in SEQ ID NO: 1, the mutations being R42L, R42I, or R42Q and V177A, V177S, or V177G. In one or more embodiments, the modified luciferase of this aspect has further enhanced luminescence compared with the wild-type luciferase (luciferase from Mycena citricolor) having the amino acid sequence shown in SEQ ID NO: 9.

In one or more embodiments, the modified luciferase of the present disclosure is an isolated polypeptide that has the amino acid sequence shown in SEQ ID NO: 11, and mutations introduced into lysine at position 27, which corresponds to position 26 of the amino acid sequence shown in SEQ ID NO: 1, and valine at position 160, which corresponds to position 161 of the amino acid sequence shown in SEQ ID NO: 1, the mutations being K27L, K27I, or K27Q and V160A, V160S, or V160G. In one or more embodiments, the modified luciferase of this aspect has further enhanced luminescence compared with the wild-type luciferase (luciferase from Neonothopanus gardneri) having the amino acid sequence shown in SEQ ID NO: 11.

In one or more embodiments, the modified luciferase of the present disclosure is an isolated polypeptide that has the amino acid sequence shown in SEQ ID NO: 13, and mutations introduced into arginine at position 36, which corresponds to position 26 of the amino acid sequence shown in SEQ ID NO: 1, and valine at position 171, which corresponds to position 161 of the amino acid sequence shown in SEQ ID NO: 1, the mutations being R36L, R36I, or R36Q and V171A, V171S, or V171G. In one or more embodiments, the modified luciferase of this aspect has further enhanced luminescence compared with the wild-type luciferase (luciferase from Omphalotus olearius) having the amino acid sequence shown in SEQ ID NO: 13.

In one or more embodiments, the modified luciferase of the present disclosure is an isolated polypeptide that has the amino acid sequence shown in SEQ ID NO: 15, and mutations introduced into arginine at position 29, which corresponds to position 26 of the amino acid sequence shown in SEQ ID NO: 1, and valine at position 171, which corresponds to position 161 of the amino acid sequence shown in SEQ ID NO: 1, the mutations being R29L, R29I, or R29Q and V171A, V171S, or V171G. In one or more embodiments, the modified luciferase of this aspect has further enhanced luminescence compared with the wild-type luciferase (luciferase from Panellus stipticus) having the amino acid sequence shown in SEQ ID NO: 15.

In one or more embodiments, the modified luciferase of the present disclosure is an isolated polypeptide that has the amino acid sequence shown in SEQ ID NO: 17, and mutations introduced into arginine at position 25, which corresponds to position 26 of the amino acid sequence shown in SEQ ID NO: 1, and valine at position 164, which corresponds to position 161 of the amino acid sequence shown in SEQ ID NO: 1, the mutations being R25L, R25I, or R25Q and V164A, V164S, or V164G. In one or more embodiments, the modified luciferase of this aspect has further enhanced luminescence compared with the wild-type luciferase (luciferase from Mycena chlorophos) having the amino acid sequence shown in SEQ ID NO: 17.

Luciferase from Neonothopanus nambi is known to maintain its luminescent function even when it is a luciferase fragment with 6, 9, 12, 15, 25, 31, 33, or 35 amino acids deleted from the N-terminus. Examples of amino acid sequences of functional fragments of luciferases from luminescent mushrooms are shown in SEQ ID NOS: 20, 21, 22, 23 24, 25, 26, 27, and 28. In one or more embodiments, a functional fragment of the modified luciferase of the present disclosure has any amino acid sequence selected from the group consisting of (d) to (0 below, and a mutation is introduced into at least one of arginine or lysine at position 5 and valine at position 140, in the amino acid sequence selected from the group consisting of (d) to (f) below.
(d) An amino acid sequence as set forth in SEQ ID NO: 20, 21, 22, 23, 24, 25, 26, 27, or 28.
(e) An amino acid sequence that is at least 80% identical to the amino acid sequence as set forth in SEQ ID NO: 20, 21, 22, 23, 24, 25, 26, 27, or 28, wherein the amino acid at position 5 is arginine or lysine, and the amino acid at position 140 is valine. In one or more embodiments, the sequence identity is at least 85%, at least 90%, at least 91%, at least 92%, at least 93%, at least 94%, at least 95%, at least 96%, at least 97%, at least 98%, or at least 99%.
(f) An amino acid sequence that has an amino acid sequence including one to several amino acid substitutions, deletions, insertions, and/or additions relative to the amino acid sequence as set forth in SEQ ID NO: 20, 21, 22, 23, 24, 25, 26, 27, or 28, wherein the amino acid at position 5 is arginine or lysine, and the amino acid at position 140 is valine. In one or more embodiments, the number of amino acid substitutions, deletions, insertions, and/or additions is 1, 2, 3, 4, 5, 6, 7, 8, 9, 10, or 11.

SEQ ID NO: 20 is an example of a functional fragment of luciferase from Mycena chlorophos, which corresponds to the amino acid sequence at positions 22 to 242 in SEQ ID NO: 1.

SEQ ID NO: 21 is an example of a functional fragment of luciferase from Neonothopanus nambi, which corresponds to the amino acid sequence at positions 36 to 256 in SEQ ID NO: 3.

SEQ ID NO: 22 is an example of a functional fragment of luciferase from Armillaria gallica, which corresponds to the amino acid sequence at positions 38 to 258 in SEQ ID NO: 5.

SEQ ID NO: 23 is an example of a functional fragment of luciferase from Armillaria mellea, which corresponds to the amino acid sequence at positions 38 to 258 in SEQ ID NO: 7.

SEQ ID NO: 24 is an example of a functional fragment of luciferase from Armillaria ostoyae, which corresponds to the amino acid sequence at positions 38 to 258 in SEQ ID NO: 9.

SEQ ID NO: 25 is an example of a functional fragment of luciferase from Mycena citricolor; which corresponds to the amino acid sequence at positions 23 to 241 in SEQ ID NO: 11.

SEQ ID NO: 26 is an example of a functional fragment of luciferase from Neonothopanus gardneri; which corresponds to the amino acid sequence at positions 32 to 252 in SEQ ID NO: 13.

SEQ ID NO: 27 is an example of a functional fragment of luciferase from Omphalotus olearius, which corresponds to the amino acid sequence at positions 25 to 245 in SEQ ID NO: 15.

SEQ ID NO: 28 is an example of a functional fragment of luciferase from Panellus stipticus, which corresponds to the amino acid sequence at positions 21 to 241 in SEQ ID NO: 17.

The modified luciferase of the present disclosure enables emission of detectable light by catalyzing an oxidation reaction of a luminescent substrate (luciferin). In one or more embodiments, an example of the luminescent substrate for the modified luciferase of the present disclosure is 3-hydroxyhispidin, which has the following structure:

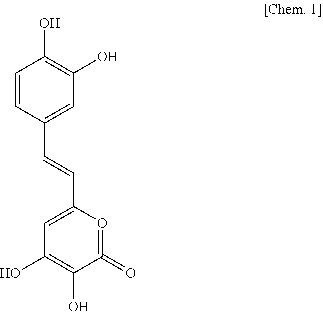

In one or more embodiments, other examples of the luminescent substrate include compounds having the following structures ((E)-6-(4-diethylamino)styryl)-3,4-dihydroxy-2H-pyran-2-one, (E)-3,4-dihydroxy-6-(4-hydroxystyryl)-2H-pyran-2-one, (E)-6-(2-1H-indol-3-yl)vinyl)-3,4-dihydroxy-2H-pyran-2-one, and (E)-6-(2-(1,2,3,5,6,7-hexahydropyrido[3,2,1-ij]quinoline-9-yl)vinyl)-3,4-dihydroxy-2H-pyran-2-one).

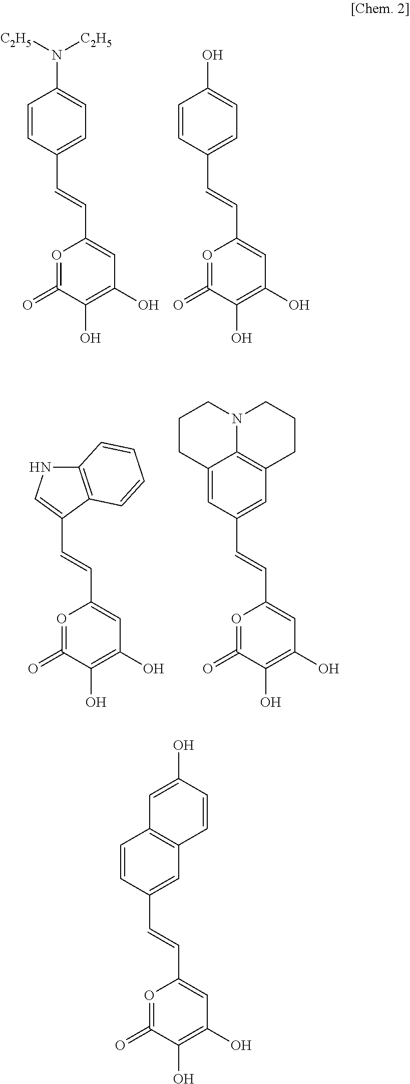

In one or more embodiments, the modified luciferase of the present disclosure may use coelenterazine as a luminescent substrate. In one or more embodiments, examples of coelenterazine may include those disclosed in U.S. patent application Ser. No. 12/056,073 (paragraph [0086]), such as, for example, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine. In addition, in one or more other embodiments, examples of “coelenterazine” may include those disclosed in WO 2012/061529. The contents of U.S. patent application Ser. No. 12/056,073 and WO 2012/061529 are incorporated herein by reference as part of the present disclosure.

In one or more embodiments, the modified luciferase of the present disclosure may be synthesized by chemical synthesis.

### Fusion Protein

In one or more embodiments, the modified luciferase of the present disclosure may be a fusion protein or fusion polypeptide having an additional amino acid at the N-terminus and/or the C-terminus. In one aspect, the present disclosure relates to a fusion protein obtained by fusing the modified luciferase of the present disclosure or a functional fragment thereof to another protein. In one or more embodiments, examples of the other protein to be fused include a fluorescent substance, an antibody, and the like. In one or more embodiments, the fusion protein of the present disclosure may be, for example, a fusion protein fused to a fluorescent polypeptide.

In one or more embodiments, the fusion protein of the present disclosure may be a fusion protein in which the modified luciferase of the present disclosure or a functional fragment thereof is linked to a fluorescent protein via a linker sequence in such a manner that enables resonance energy transfer from the modified luciferase or functional fragment thereof to the fluorescent protein. Resonance energy transfer here may be bioluminescence resonance energy transfer (BRET). In one or more non-limiting embodiments, the fusion protein of the present disclosure may have the modified luciferase of the present disclosure or a functional fragment thereof on the N-terminal side of the fusion protein and a fluorescent substance on the C-terminal side.

In one or more embodiments, the fusion protein of the present disclosure may be synthesized by chemical synthesis or may be produced using genetic recombination technology.

### Isolated Nucleic Acid (Nucleic Acid Molecule)

In one aspect, the present disclosure relates to an isolated nucleic acid encoding the modified luciferase of the present disclosure or a functional fragment thereof.

In one or more embodiments, the nucleic acid of the present disclosure encodes a modified luciferase or a functional fragment thereof that includes a mutation introduced into arginine or lysine corresponding to position 26 of the amino acid sequence shown in SEQ ID NO: 1 and/or a mutation introduced into valine corresponding to position 161 of the amino acid sequence shown in SEQ ID NO: 1 and that has enhanced luminescence compared with a luciferase from a luminescent mushroom without or before the introduction of the mutation or a wild-type luciferase derived from a luminescent mushroom.

In one or more embodiments, the nucleic acid of the present disclosure relates to an isolated nucleic acid encoding a polypeptide or a functional fragment thereof that has any amino acid sequence selected from the group consisting of (a) to (c) below, and a mutation introduced into arginine or lysine corresponding to position 26 of the amino acid sequence shown in SEQ ID NO: 1 and/or a mutation valine corresponding to position 161 of the amino acid sequence shown in SEQ ID NO: 1, the amino acid sequence selected from the group consisting of (a) to (c) below.
(a) An amino acid sequence of a luciferase derived from a luminescent mushroom, which is set forth in SEQ ID NO: 1, 3, 5, 7, 9, 11, 13, 15, or 17.
(b) An amino acid sequence that is at least 80% identical to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine. In one or more embodiments, the sequence identity is at least 85%, at least 90%, at least 91%, at least 92%, at least 93%, at least 94%, at least 95%, at least 96%, at least 97%, at least 98%, or at least 99%.
(c) An amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine. In one or more embodiments, the number of amino acid substitutions, deletions, insertions, and/or additions is 1, 2, 3, 4, 5, 6, 7, 8, 9, 10, or 11.

In one or more embodiments, the nucleic acid of the present disclosure relates to an isolated nucleic acid encoding a functional fragment of the modified luciferase of the present disclosure that has any amino acid sequence selected from the group consisting of (d) to (f) below and a mutation introduced into arginine or lysine at position 5 and/or a mutation introduced into valine at position 140, in the amino acid sequence selected from the group consisting of (d) to (f) below.
(d) An amino acid sequence as set forth in SEQ ID NO: 20, 21, 22, 23, 24, 25, 26, 27, or 28.
(e) An amino acid sequence that is at least 80% identical to the amino acid sequence as set forth in SEQ ID NO: 20, 21, 22, 23, 24, 25, 26, 27, or 28, wherein the amino acid at position 5 is arginine or lysine, and the amino acid at position 140 is valine. In one or more embodiments, the sequence identity is at least 85%, at least 90%, at least 91%, at least 92%, at least 93%, at least 94%, at least 95%, at least 96%, at least 97%, at least 98%, or at least 99%.
(f) An amino acid sequence that has an amino acid sequence including one to several amino acid substitutions, deletions, insertions, and/or additions relative to the amino acid sequence as set forth in SEQ ID NO: 20, 21, 22, 23, 24, 25, 26, 27, or 28, wherein the amino acid at position 5 is arginine or lysine, and the amino acid at position 140 is valine. In one or more embodiments, the number of amino acid substitutions, deletions, insertions, and/or additions is 1, 2, 3, 4, 5, 6, 7, 8, 9, 10, or 11.

In one or more embodiments, the nucleic acid of the present disclosure encodes a modified luciferase obtained by modifying luciferase from Mycena chlorophos(SEQ ID NO: 1), luciferase from Neonothopanus nambi(SEQ ID NO: 3), luciferase from Armillaria gallica (SEQ ID NO: 5), luciferase from Armillaria mellea (SEQ ID NO: 7), luciferase from Armillaria ostoyae (SEQ ID NO: 9), luciferase from Mycena citricolor (SEQ ID NO: 11), luciferase from Neonothopanus gardneri(SEQ ID NO: 13), luciferase from Omphalotus olearius (SEQ ID NO: 15), or luciferase from Panellus stipticus (SEQ ID NO: 17), wherein a mutation is introduced into arginine or lysine corresponding to position 26 of the amino acid sequence shown in SEQ ID NO: 1 and/or a mutation is introduced into valine corresponding to position 161 of the amino acid sequence shown in SEQ ID NO: 1.

In one or more embodiments, in order to achieve even higher luminescence intensity the nucleic acid of the present disclosure encodes a modified luciferase obtained by modifying luciferase from Mycena chlorophos(SEQ ID NO: 1), luciferase from Neonothopanus nambi (SEQ ID NO: 3), luciferase from Armillaria gallica (SEQ ID NO: 5), luciferase from Armillaria mellea (SEQ ID NO: 7), luciferase from Armillaria ostoyae (SEQ ID NO: 9), luciferase from Mycena citricolor (SEQ ID NO: 11), luciferase from Neonothopanus gardneri (SEQ ID NO: 13), luciferase from Omphalotus olearius (SEQ ID NO: 15), or luciferase from Panellus stipticus (SEQ ID NO: 17), wherein mutations are introduced into both the arginine or lysine corresponding to position 26 and the valine corresponding to position 161 of the amino acid sequence shown in SEQ ID NO: 1.

In one or more embodiments, a DNA base sequence encoding the luciferase shown in SEQ ID NO: 1 may be the base sequence shown in SEQ ID NO: 2. In one or more embodiments, a DNA base sequence encoding the luciferase shown in SEQ ID NO: 3 may be the base sequence shown in SEQ ID NO: 4. In one or more embodiments, a DNAbase sequence encoding the luciferase shown in SEQ ID NO: 5 may be the base sequence shown in SEQ ID NO: 6. In one or more embodiments, a DNAbase sequence encoding the luciferase shown in SEQ ID NO: 7 may be the base sequence shown in SEQ ID NO: 8. In one or more embodiments, a DNA base sequence encoding the luciferase shown in SEQ ID NO: 9 may be the base sequence shown in SEQ ID NO: 10. In one or more embodiments, a DNAbase sequence encoding the luciferase shown in SEQ ID NO: 11 may be the base sequence shown in SEQ ID NO: 12. In one or more embodiments, a DNAbase sequence encoding the luciferase shown in SEQ ID NO: 13 may be the base sequence shown in SEQ ID NO: 14. In one or more embodiments, a DNAbase sequence encoding the luciferase shown in SEQ ID NO: 15 may be the base sequence shown in SEQ ID NO: 16. In one or more embodiments, a DNA base sequence encoding the luciferase shown in SEQ ID NO: 17 may be the base sequence shown in SEQ ID NO: 18.

In one or more non-limiting embodiments, a nucleic acid that contains a base sequence encoding the modified luciferase of the present disclosure can be easily obtained by introducing a mutation into the base sequence shown in SEQ ID NO: 2, 4, 6, 8, 10, 12, 14, 16, or 18.

In one or more embodiments, the nucleic acid of the present disclosure relates to an isolated nucleic acid encoding a polypeptide or a functional fragment thereof that has the amino acid sequence shown in SEQ ID NO: 19 or 29, and a mutation introduced into arginine or lysine at position 5 and/or a mutation introduced into valine at position 140 in the amino acid sequence shown in SEQ ID NO: 19 or 29.

In one or more embodiments, the nucleic acid of the present disclosure may include a nucleic acid encoding the fusion protein of the present disclosure.

### Expression Cassette

In one aspect, the present disclosure relates to an expression cassette used to obtain the modified luciferase of the present disclosure or the fusion protein of the present disclosure or to replicate the nucleic acid of the present disclosure. In one or more embodiments, the expression cassette may be present as an extrachromosomal element, or may be integrated into the genome of a cell by introducing the expression cassette into the cell.

Thus, in one or more embodiments, the expression cassette of the present disclosure relates to an expression cassette that is under the control of a regulatory element required for the expression of a nucleic acid in a host cell and that contains the nucleic acid of the present disclosure, the expression cassette being integrated into the genome of a cell or introduced into the cell in the form of an extrachromosomal element and being capable of causing the expression of a luciferase encoded by the nucleic acid of the present disclosure.

### Transformant

In one or more embodiments, the nucleic acid and expression cassette of the present disclosure can be used to produce a transformant. In one or more embodiments, the transformant of the present disclosure contains the nucleic acid of the present disclosure introduced by transfection.

As the method for obtaining the transformant, previously reported and established methods can be used as appropriate. In one or more embodiments, examples of the method for obtaining the transformant include an Agrobacterium method, a PEG-calcium phosphate method, an electroporation method, a liposome method, a particle gun method, a microinjection method, and the like.

In the Agrobacterium method, a protoplast may be used, a tissue fragment may be used, or the plant body itself may be used (in planta method). When a protoplast is used, the method can be performed, for example, by co-culturing the protoplast with an Agrobacterium having a Ti plasmid or by fusing the protoplast to a spheroplasted Agrobacterium (spheroplast method). When a tissue fragment is used, the method can be performed, for example, by infecting a sterile-cultured leaf piece (leaf disc) of a target plant or by infecting a callus. When the in planta method, which uses a seed or a plant body, is applied, or in other words, in a system that does not involve tissue culture supplemented with a plant hormone, the method can be performed, for example, by directly treating a water-absorbing seed, a young plant (seedling), a potted plant, or the like with Agrobacterium.

In one aspect, the present
disclosure relates to a cell that produces a luciferase encoded by the nucleic acid of the present disclosure. This cell relates to a cell containing an expression cassette that is under the control of a regulatory element required for the expression of a nucleic acid in a host cell and that contains the nucleic acid of the present disclosure, the expression cassette being integrated into the genome of the cell or introduced into the cell in the form of an extrachromosomal element, and being capable of causing the expression of a luciferase encoded by the nucleic acid of the present disclosure, in the form of the extrachromosomal element or an element integrated into the genome of the cell.

In one or more embodiments, a prokaryotic cell and a eukaryotic cell can be used as the host. Examples of the host cell include Escherichia cob, Bacillus subtilis, yeast, plants, insect cells, non-human vertebrate cells, and the like. Examples of the plants include Arabidopsis thaliana, Nicotiana benthamiana, Physcomitrella patens, and the like.

### Transgenic Organism

In one aspect, the present disclosure relates to a transgenic organism containing the nucleic acid of the present disclosure. In one or more embodiments, examples of the transgenic organism include transgenic plants and transgenic animals.

In one or more embodiments, examples of the transgenic plants include transgenics of plants such as Arabidopsis thaliana, Nicotiana benthamiana, Physcomitrella patens, Brachypodium distachyon, rice, wheat, and barley. In one or more embodiments, examples of the transgenic animals include transgenics of non-human animals such as zebrafish, mice, rats, and pigs.

In one or more embodiments, the transgenic organism of the present disclosure can be produced by introducing the nucleic acid of the present disclosure, the modified luciferase of the present disclosure, the expression cassette of the present disclosure, or a vector of the present disclosure into a subject (organism). In one or more embodiments, the method for producing the transgenic organism can be determined as appropriate according to the type of the transgenic organism. In one or more embodiments, examples of the method for producing a transgenic plant include an Agrobacterium method, a PEG-calcium phosphate method, an electroporation method, a liposome method, a particle gun method, a microinjection method, and the like. In one or more embodiments, examples of the method for producing a transgenic animal include a method in which the nucleic acid of the present disclosure is introduced into a fertilized egg collected from an organism, a method in which a cell from the early developing lung derived from a donor organism is infected ex vivo with a viral vector such as a retrovirus, and the like.

In one or more embodiments, a group of genes constituting the biosynthetic cycle of a luminescent substrate, such as 3-hydroxyhispidin, may be further introduced into the transgenic organism of the present disclosure. With the transgenic organism of this aspect, in one or more embodiments, autonomous luminescence imaging (autoluminescence imaging) that performs imaging can be performed.

### Kit

In one aspect, the present disclosure relates to a kit including the modified luciferase of the present disclosure, the nucleic acid encoding the modified luciferase of the present disclosure, the expression cassette of the present disclosure, and/or the fusion protein of the present disclosure. In one or more embodiments, the kit of the present disclosure may further include a luminescent substrate (luciferin) of the modified luciferase of the present disclosure. The luminescent substrate (luciferin) is as described above.

In one or more embodiments, constituent components of the kit may be contained in a container. In one or more embodiments, the constituent components of the kit may be stored in a dry state, or may be stored in a medium such as a buffer.

In one or more embodiments, the kit of the present disclosure may include instructions for use of each constituent component.

### Applications

The modified luciferase, fusion protein, nucleic acid, expression cassette, and kit of the present disclosure can be used in various fields, including medicine, biotechnology, and the like. In one or more embodiments, an example of their applications is autoluminescence-based imaging. In one or more embodiments, other examples of their applications include analysis of intracellular functions, diagnostics, quality control, environmental testing, and the like. In one or more embodiments, yet other examples of their applications include the production of bioluminescent transgenic plants that can be used as light sources for illumination and other similar purposes.

In another aspect, the present disclosure relates to a method for causing the expression of the modified luciferase of the present disclosure or a functional fragment thereof. In one or more embodiments, the method of this aspect includes introducing the nucleic acid of the present disclosure into a cell or plant and causing an oxidation reaction of a luminescent substrate in the introduced cell or plant.

In another aspect, the present disclosure relates to a method for producing bioluminescence. In one or more embodiments, the method of this aspect includes adding a luminescent substrate to a medium or soil in which a transgenic plant having the nucleic acid of the present disclosure is placed.

In one or more embodiments, the present disclosure relates to use of a transgenic plant having the nucleic acid of the present disclosure and a group of genes constituting the biosynthetic cycle of a luminescent substrate.

The present disclosure further relates to one or more non-limiting embodiments below.

[1] A modified luciferase derived from a luminescent mushroom or a functional fragment thereof, including a mutation introduced into arginine or lysine corresponding to position 26 of amino acid sequence shown in SEQ ID NO: 1 and/or a mutation introduced into valine corresponding to position 161 of amino acid sequence shown in SEQ ID NO: 1, and having enhanced luminescence compared with a luciferase before the mutation is introduced.

[2] The modified luciferase or functional fragment thereof according to [1], w herein the modified luciferase is a variant obtained by modifying a luciferase from a luminescent mushroom having an amino acid sequence as set forth in SEQ ID NO: 19: FPXXXXDYXTFLXXGPSYAPQNXXGYXIVXVLXLFRXEXXXXXIYXXX PEKRXWLXXLPXRXGXRPXXTSHIIQRQXXQXXDXXFXXXXXXXXIXRXQXRHX XXTXDXXSXFEFHAXAIFXXXXXXXXXPXXXPXXXTVRRTKXEIAHMHDYHDXTX HLALAAXDXKXVXXKGWGQRHPLAGPGXPGPPXEWTFXYAPRXEXEXXVXEXI XEAXXXYMIXN or an amino acid sequence as set forth in SEQ ID NO: 29: FP XXXXDYXTFLXXGPSYAPQNXXGYXIVXVLXLFRXEXXXXXIYXXXPEKRXWLXX LPXRXGXRPXXTSHIIQRQXXQXXDXXFXXXXLXXXXIXRXQXRHXXXTXXXXSXF EFHAXAIFXXXXXXXXXPXXXPXXXTVRRTKXEIAHMHDYHDXTXHLALAAXDX KXVXXKGWGQRHPLAGPGXPGPPXEWTFXYAPRXEXEXXVXEXIXEAXXXYMX N.

[3] An isolated nucleic acid encoding the modified luciferase or functional fragment thereof according to [1] or [2].

[4] An isolated nucleic acid encoding a modified luciferase or a functional fragment thereof having any amino acid sequence selected from the group consisting of (a) to (c) below, and a mutation introduced into arginine or lysine corresponding to position 26 of amino acid sequence shown in SEQ ID NO:1 and/or a mutation introduced into valine corresponding to position 161 of amino acid sequence shown in SEQ ID NO: 1 in the amino acid sequence selected from the group consisting of (a) to (c) below:
(a) an amino acid sequence of a luciferase derived from a luminescent mushroom, which is set forth in SEQ ID NO: 1, 3, 5, 7, 9, 11, 13, 15, or 17;
(b) an amino acid sequence that is at least 80% identical to the amino acid sequence set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine; and
(c) an amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine.

[5] The isolated nucleic acid according to [3] or [4], wherein the modified luciferase has enhanced luminescence compared with a luciferase before the mutation is introduced.

[6] An expression cassette under the control of a regulatory element required for the expression of a nucleic acid in a host cell, containing the nucleic acid according to any one of [3] to [5],
wherein the expression cassette is integrated into a genome of a cell or introduced into the cell in the form of an extrachromosomal element, and
the expression cassette is capable of causing the expression of a luciferase encoded by the nucleic acid according to any one of [3] to [5].

[7] A cell producing a luciferase encoded by the nucleic acid according to any one of [3] to [5], containing:
an expression cassette under the control of a regulatory element required for the expression of a nucleic acid in a host cell, the expression cassette containing the nucleic acid according to any one of [3] to [5],
wherein the expression cassette is integrated into a genome of the cell or introduced into the cell in the form of an extrachromosomal element, and
the expression cassette in the form of the extrachromosomal element or an element integrated into the genome of the cell is capable of causing the expression of the luciferase encoded by the nucleic acid according to any one of [3] to [5].

[8] A modified luciferase or a functional fragment thereof having any amino acid sequence selected from the group consisting of (a) to (c) below, and a mutation introduced into arginine or lysine corresponding to position 26 of the amino acid sequence shown in SEQ ID NO:1 and/or a mutation introduced into valine corresponding to position 161 of amino acid sequence shown in SEQ ID NO: 1, in the amino acid sequence selected from the group consisting of (a) to (c) below:
(a) an amino acid sequence of a luciferase derived from a luminescent mushroom, which is set forth in SEQ ID NO: 1, 3, 5, 7, 9, 11, 13, 15, or 17;
(b) an amino acid sequence that is at least 80% identical to the amino acid sequence set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine; and
(c) an amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine.

[9] The modified luciferase or functional fragment thereof according to [8], wherein the modified luciferase has enhanced luminescence compared with a luciferase before the mutation is introduced.

[10] A fusion protein obtained by fusing the modified luciferase or functional fragment thereof according to [1], [2], [8], or [9] to another protein.

[11] A fusion protein, wherein the modified luciferase or functional fragment thereof according to [1], [2], [8], or [9] is linked to a fluorescent protein via a linker sequence in such a manner that enables resonance energy transfer from the modified luciferase or functional fragment thereof to the fluorescent protein.

[12] An isolated nucleic acid encoding the fusion protein according to [10] or [11].

[13] A kit including at least one selected from the group consisting of (1) to (6) below and a luminescent substrate for the modified luciferase according to [1], [2], [8], or [9]:
(1) the isolated nucleic acid according to any one of [3] to [5] and [12];
(2) the isolated luciferase or functional fragment thereof according to [1], [2], [8], or [9];
(3) an expression cassette containing the nucleic acid according to any one of [3] to [5] and [12];
(4) the expression cassette according to [6];
(5) a fusion protein obtained by fusing the modified luciferase or functional fragment thereof according to [1], [2], [8], or [9] to another protein; and
(6) the fusion protein according to [10] or [11].

[14] The kit according to [13], wherein the luminescent substrate is selected from the group consisting of 3-hydroxyhispidin, (E)-6-(4-diethylamino)styryl)-3,4-dihydroxy-2H-pyran-2-one, (E)-3,4-dihydroxy-6-(4-hydroxystyryl)-2H-pyran-2-one, (E)-6-(2-1H-indol-3-yl)vinyl)-3,4-dihydroxy-2H-pyran-2-one, (E)-6-(2-(1,2,3,5,6,7-hexahydropyrido[3,2,1-ij]quinoline-9-yl)vinyl)-3,4-dihydroxy-2H-pyran-2-one), coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v and 2-methyl coelenterazine.

[15] A transgenic organism containing the nucleic acid according to any one of [3] to [5] and [12].

Hereinafter, the present disclosure will be described in greater detail by means of examples; however, the examples below should be construed as illustrative, and the present disclosure is not limited to these examples.

### EXAMPLES

### Example 1: Production of Mutated Luciferase 1

A variant cDNA library was produced by inducing random mutations into the full-length gene sequence (SEQ ID NO: 2) of the luciferase from Mycena chlorophos. Escherichia coli (JM109 (DE3)) transformed with a plasmid containing the above gene was cultured (37° C./16 hours) in an LB agar medium containing 100 μg/mL ampicillin to express the protein of interest, and E. coli colonies were screened by obtaining a luminescence image under the conditions supplemented with 3-hydroxyhispidin.

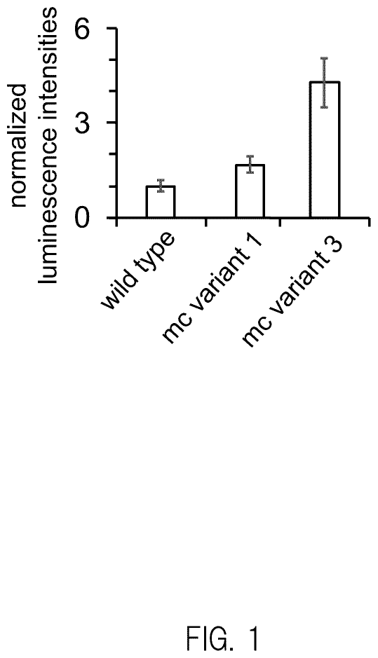

As a result, the following variants, which showed higher expression levels than the wild type, were obtained in the E. coli colonies. FIG. 1 shows examples of the results. The locations of the mutations indicated below correspond to the positions of amino acids in the amino acid sequence (SEQ ID NO: 1) of luciferase from Mycena chlorophos.
me Variant 1 (mutation: R26L)
me Variant 2 (mutation: V161A)
me Variant 3 (mutations: R26L and V161A)

During the screening of the E. coli colonies, the luminescence intensity was measured using the following procedure. E. coli (JM109 (DE3)) transformed with a plasmid designed to express the protein of interest was cultured (37° C./16 hours) in an LB agar medium containing 100 μg/mL ampicillin. A replica was produced using a membrane as in the screening, and then, the protein was expressed in a medium containing IPTG. The membrane was immersed in 5 mL of buffer (PBS, pH 8.0). Then, the membrane was transferred to an empty 10-cm dish, and 5 mL of a 10 μM 3-hydroxyhispidin solution (10 μM 3-hydroxyhispidin; PBS, pH 8.0) was added. This was imaged using LAS-3000 (Fujifilm) (observation conditions: sensitivity: High, exposure time: 5 minutes). After background noise was removed from the obtained luminescence image using ImageJ software, the image was identically duplicated. For one of the images, signals were normalized using the Threshold function, and regions where fluorescence intensity was to be automatically examined (regions of interest: ROIs) were set. In the other image (duplicated image), signals in each of the set ROIs were measured. For the Threshold function, wild-type mcLuz (luciferase from Mycena chlorophos) was used. FIG. 1 shows the results.

FIG. 1 shows normalized luminescence intensities of the wild type and the me variants 1 and 3 in the Escherichia coli colonies. The luminescence intensity of the me variant 1 was at least 1.5 times higher than that of the wild type, and the luminescence intensity of the me variant 3 was at least 4 times higher than that of the wild type. In addition, the mutation V161A was found to cause a change in luminescence intensity in conjunction with the mutation R26L.

### Example 2: Multiple Alignment of Amino Acid Sequences of Various Luciferases

To determine whether the mutation positions in Example 1 also affect the luminescence intensity in luciferases from other luminescent mushrooms, a multiple alignment of the amino acid sequences of nine previously identified luciferases from luminescent mushrooms was performed using GENETYX Ver. 12 (GENETYX).

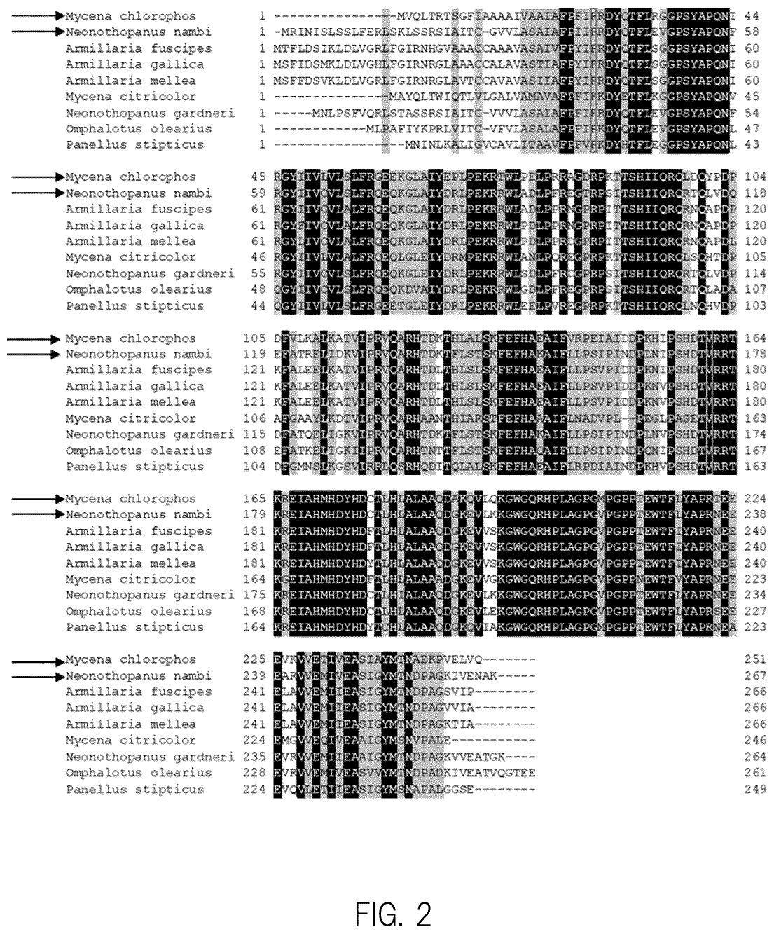

The amino acid sequences of the nine types of Luz (luciferase) were obtained using NCBI (https://www.ncbi.nlm.nih.gov/). FIG. 2 shows the results. Their respective GenBank IDs are as follows: luciferase from Mycena chlorophos (GenBank: BBH43507.1, SEQ ID NO: 1), luciferase from Neonothopanus nambi (GenBank: BBH43509.1, SEQ ID NO: 3), luciferase from Armillaria gallica (GenBank: BBH43503.1, SEQ ID NO: 5), luciferase from Armillaria mellea (GenBank: BBH43504.1, SEQ ID NO: 7), luciferase from Armillaria ostoyae (GenBank: BBH43505.1, SEQ ID NO: 9), luciferase from Mycena citricolor(GenBank: BBH43506.1, SEQ ID NO: 11), luciferase from Neonothopanus gardneri(GenBank: BBH43508.1, SEQ ID NO: 13), luciferase from Omphalotus olearius(GenBank: BBH43510.1, SEQ ID NO: 15), and luciferase from Panellus stipticus (GenBank: BBH43513.1, SEQ ID NO: 17).

In the nine luciferases from the luminescent mushrooms listed above, arginine at position 26 of luciferase from Mycena chlorophos, which was mutated in Example 1, was conserved in eight of the nine types, and valine at position 161 thereof was conserved in all nine types.

### Example 3: Production of Mutated Luciferase 2

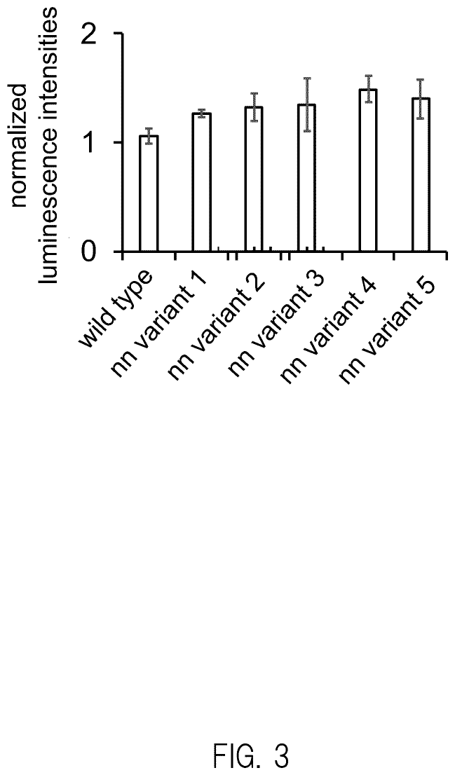

As a result of the alignment in Example 2, it was predicted that arginine at position 26 and valine at position 161 of luciferase from Mycena chlorophos correspond to arginine at position 40 and valine at position 175, respectively, of luciferase from Neonothopanus nambi Based on this result, the following variants were produced in the same manner as in Example 1, in which arginine at position 40 and valine at position 175 of luciferase from Neonothopanus nambi(SEQ ID NO: 3) were mutated. FIG. 3 shows examples of the results. The locations of the mutations indicated below correspond to the positions in the amino acid sequence (SEQ ID NO: 3) of luciferase from Neonothopanus nambi
nn Variant 1 (mutation: R40I)
nn Variant 2 (mutations: R40I and V175S)
nn Variant 3 (mutation: R40Q)
nn Variant 4 (mutations: R40Q and V175A)
nn Variant 5 (mutations: R40Q and V175G)

FIG. 3 shows normalized luminescence intensities of the wild type and the nn variants 1 to 5 in the E. coli colonies. The luminescence intensities of all these variants were at least about 1.5 times higher than that of the wild type.

SEQUENCE
Mycena chlorophos_amino acid sequence
Genbank ID: BBH43507
SEQ ID NO: 1
MVQLTRTSGFIAAAAIVAAIAFPFIRRDYQTFLRGGPSYAPQNIRGYIIV
LVLSLFRGEEKGLAIYEPLPEKRTWLPELPRRAGDRPKTTSHIIQRQLDQ
YPDPDFVLKALKATVIPRVQARHTDKTHLALSKFEFHAEAIFVRPEIAID
DPKHIPSHDTVRRTKREIAHMHDYHDCTLHLALAAQDAKQVLQKGWGQRH
PLAGPGMPGPPTEWTFLYAPRTEEEVKVVETIVEASIAYMTNAEKPVELV
Q
Mycena chlorophos_nucleotide sequence
Genbank ID: LC435377
SEQ ID NO: 2
ATGGTCCAACTCACCCGAACCTCCGGATTCATCGCCGCTGCGGCCATTGT
TGCTGCCATCGCCTTCCCGTTCATTCGTCGAGACTACCAGACGTTCCTTC
GTGGTGGGCCGTCCTATGCCCCACAGAACATCCGCGGCTATATCATCGTC
CTGGTTCTGTCCCTCTTCCGCGGCGAGGAGAAGGGTCTTGCAATCTACGA
GCCCCTTCCTGAGAAGCGCACATGGCTGCCGGAGCTTCCGCGGCGCGCGG
GAGACCGGCCCAAGACGACGAGCCACATCATCCAACGGCAGCTCGACCAG
TACCCCGACCCGGACTTTGTCCTCAAAGCCCTGAAAGCGACGGTCATCCC
GCGTGTCCAAGCCCGGCACACAGACAAGACTCACCTCGCGCTGTCCAAGT
TCGAGTTCCATGCTGAGGCCATCTTCGTGCGCCCGGAAATCGCCATCGAC
GACCCGAAGCATATCCCCAGCCACGACACGGTGCGACGGACGAAGCGCGA
GATTGCGCACATGCACGACTATCACGACTGCACGCTGCATTTGGCGCTAG
CGGCGCAGGACGCGAAGCAGGTGCTGCAGAAGGGCTGGGGCCAGCGCCAT
CCGCTGGCAGGGCCTGGGATGCCCGGGCCGCCCACGGAGTGGACGTTCTT
GTATGCCCCGAGGACCGAGGAGGAAGTGAAGGTTGTGGAGACCATTGTCG
AGGCCTCTATCGCGTACATGACGAACGCGGAGAAGCCGGTCGAGCTGGTG
CAG
Neonothopanus namb_amino acid sequence
Genbank ID:
SEQ ID NO: 3
MRINISLSSLFERLSKLSSRSIAITCGVVLASAIAFPIIRRDYQTFLEVG
PSYAPQNFRGYIIVCVLSLFRQEQKGLAIYDRLPEKRRWLADLPFREGTR
PSITSHIIQRQRTQLVDQEFATRELIDKVIPRVQARHTDKTFLSTSKFEF
HAKAIFLLPSIPINDPLNIPSHDTVRRTKREIAHMHDYHDCTLHLALAAQ
DGKEVLKKGWGQRHPLAGPGVPGPPTEWTFLYAPRNEEEARVVEMIVEAS
IGYMTNDPAGKIVENAK
Neonothopanus namb_nucleotide sequence
Genbank ID: LC435379
SEQ ID NO: 4
ATGCGCATTAACATTAGCCTCTCGTCTCTCTTCGAACGTCTCTCCAAACT
TAGCAGTCGCAGCATAGCGATTACATGTGGAGTTGTTCTCGCCTCCGCAA
TCGCCTTTCCCATCATCCGCAGAGACTACCAGACTTTCCTAGAAGTGGGA
CCCTCGTACGCTCCGCAGAACTTTAGAGGATACATCATCGTCTGTGTCCT
CTCGCTATTCCGCCAAGAGCAGAAAGGGCTCGCCATCTATGATCGTCTTC
CCGAGAAACGCAGGTGGTTGGCCGACCTTCCCTTTCGTGAAGGAACCAGA
CCCAGCATTACCAGCCATATCATTCAGCGACAGCGCACTCAACTGGTCGA
TCAGGAGTTTGCCACCAGGGAGCTCATAGACAAGGTCATCCCTCGCGTGC
AAGCACGACACACCGACAAAACGTTCCTCAGCACATCAAAGTTCGAGTTT
CATGCGAAGGCCATATTTCTCTTGCCTTCTATCCCAATCAACGACCCTCT
GAATATCCCTAGCCACGACACTGTCCGCCGAACGAAGCGCGAGATTGCAC
ATATGCATGATTATCATGATTGCACACTTCATCTTGCTCTCGCTGCGCAG
GATGGAAAGGAGGTGCTGAAGAAAGGTTGGGGACAACGACATCCTTTGGC
TGGTCCTGGAGTTCCTGGTCCACCAACGGAATGGACTTTTCTTTATGCGC
CTCGCAACGAAGAAGAGGCTCGAGTAGTGGAGATGATCGTTGAGGCTTCC
ATAGGGTATATGACGAACGATCCTGCAGGAAAGATTGTAGAAAACGCCAA
G
Armillaria gallica_amino acid sequence
Genbank ID: PBL02715
SEQ ID NO: 5
MSFIDSMKLDLVGHLFGIRNRGLAAACCALAVASTIAFPYIRRDYQTFLS
GGPSYAPQNIRGYFIVCVLALFRQEQKGLAIYDRLPEKRRWLPDLPPRNG
PRPITTSHIIQRQRNQAPDPKFALEELKATVIPRVQARHTDLTHLSLSKF
EFHAEAIFLLPSVPIDDPKNVPSHDTVRRTKREIAHMHDYHDFTLHLALA
AQDGKEVVSKGWGQRHPLAGPGVPGPPTEWTFIYAPRNEEELAVVEMIIE
ASIGYMTNDPAGVVIA
Armillaria gallica_nucleotide sequence
Genbank ID: LC435373
SEQ ID NO: 6
ATGTCCTTCATCGACAGCATGAAACTTGACCTCGTCGGACACCTCTTTGG
CATCAGGAATCGCGGCTTAGCCGCCGCTTGTTGTGCTCTAGCAGTCGCCT
CTACTATCGCCTTCCCTTACATTCGTAGGGACTACCAGACATTTTTATCT
GGCGGTCCCTCTTACGCTCCCCAGAATATCAGAGGATATTTCATCGTCTG
CGTTCTGGCCTTGTTCCGTCAGGAGCAAAAGGGCCTTGCGATATATGATC
GCCTTCCCGAGAAGCGCAGGTGGCTGCCTGACTTGCCTCCTCGCAATGGC
CCGCGGCCGATCACGACCAGCCATATAATCCAAAGACAGCGCAACCAGGC
GCCGGACCCCAAGTTCGCCCTCGAGGAACTCAAGGCCACGGTTATTCCAC
GGGTGCAGGCTCGCCATACTGACCTCACCCATCTCAGCCTATCCAAATTC
GAGTTCCATGCTGAAGCAATTTTCCTGCTCCCCTCTGTACCCATCGATGA
TCCAAAAAATGTTCCAAGTCACGACACGGTGCGCAGGACGAAAAGGGAGA
TCGCGCATATGCACGACTACCATGACTTCACGCTGCATCTTGCACTGGCC
GCCCAAGACGGGAAGGAAGTCGTGTCGAAGGGATGGGGGCAGCGACACCC
CCTAGCAGGCCCTGGCGTTCCTGGTCCACCTACGGAGTGGACATTTATTT
ATGCGCCACGTAACGAAGAGGAACTGGCAGTGGTGGAAATGATTATCGAG
GCATCAATAGGCTATATGACCAATGACCCTGCTGGAGTAGTTATCGCA
Armillaria mellea_amino acid sequence
Genbank ID: BBH43504
SEQ ID NO: 7
MSFFDSVKLDLVGRLFGIRNRGLAVTCCAVAVASIIAFPYIRRDYQTFLS
GGPSYAPQNIRGYLIVCVLALFRQEQKGLAIYDRLPEKRRWLPDLPPRDG
PRPITTSHIIQRQRNQAPDLKFALEELKATVIPRVQARHTDLTHLSLSKF
EFHAEAIFLLPSVPIDDPKNVPSHDTVRRTKREIAHMHDYHDYTLHLALA
AQDGKEVVSKGWGQRHPLAGPGVPGPPTEWTFIYAPRNEEELAVVEMIIE
ASIGYMTNDPAGKTIA
Armillaria mellea_nucleotide sequence
Genbank ID: LC435374
SEQ ID NO: 8
ATGTCCTTCTTCGACAGCGTGAAACTTGACCTCGTCGGACGCCTCTTTGG
CATCAGGAATCGCGGCTTAGCTGTTACTTGTTGTGCTGTGGCAGTCGCCT
CTATCATCGCGTTCCCTTACATTCGTAGGGACTACCAGACATTTTTATCT
GGGGGTCCCTCCTACGCTCCCCAGAACATCAGAGGATACCTCATTGTCTG
CGTCCTGGCCTTGTTCCGTCAGGAGCAAAAAGGCCTTGCGATATACGACC
GCCTTCCCGAGAAGCGCAGGTGGCTACCTGACTTGCCTCCTCGCGATGGC
CCACGGCCCATCACGACCAGCCATATAATCCAAAGACAGCGCAACCAGGC
GCCGGACCTCAAGTTCGCCCTCGAGGAACTCAAGGCCACGGTCATTCCAC
GGGTGCAGGCTCGCCACACTGACCTCACCCATCTCAGCCTATCCAAGTTC
GAGTTCCATGCTGAAGCAATCTTCCTGCTCCCCTCTGTACCCATCGATGA
TCCAAAGAATGTGCCAAGTCACGACACGGTGCGCAGGACGAAGAGGGAAA
TTGCGCATATGCACGACTACCATGACTACACGCTGCATCTTGCGTTGGCC
GCCCAAGACGGGAAGGAAGTCGTATCAAAGGGATGGGGGCAGCGACACCC
GCTGGCAGGCCCTGGCGTTCCTGGTCCACCGACGGAGTGGACGTTTATTT
ATGCGCCACGTAACGAAGAGGAGCTGGCAGTGGTGGAAATGATTATCGAG
GCATCGATAGGCTATATGACCAATGACCCTGCAGGAAAAACTATCGCA
Armillaria ostoyae_amino acid sequence
Genbank ID: BBH43505
SEQ ID NO: 9
MSFIDSMKLDFVGHLFGIRNRGLATACCAVAVASAIAFPYIRRDYQTFLS
GGPSYAPQNIKGYLIVCVLALFRQEQKGLAIYDRLPEKRRWLPDLPPRNG
PRPITTSHIIQRQRNQAPDSKFALEELKATVIPRVQARHTDLTHLSLSKF
EFHAEAIFLLPSVPIDDPKNVPSHDTVRRTKREIAHMHDYHDFTLHLALA
AQDGKEVVAKGWGQRHPLAGPGVPGPPTEWTFIYAPRNEEELAVVEMIIE
ASIGYMTNDPAGTVIV
Armillaria ostoyae_nucleotide sequence
Genbank ID: LC435375
SEQ ID NO: 10
ATGTCCTTCATCGACAGCATGAAACTTGACTTCGTCGGACACCTCTTTGG
CATCAGGAATCGCGGCTTAGCCACCGCTTGTTGTGCTGTGGCAGTCGCTT
CTGCCATCGCCTTCCCTTACATTCGTAGGGACTACCAGACATTCTTATCT
GGCGGTCCCTCTTACGCTCCCCAGAACATCAAAGGATATCTCATCGTCTG
CGTCCTGGCCTTGTTCCGTCAGGAGCAAAAGGGCCTTGCGATATATGACC
GCCTTCCCGAGAAGCGCAGGTGGCTACCTGACTTGCCTCCTCGCAATGGC
CCGCGGCCCATCACGACCAGCCATATAATCCAAAGACAGCGCAACCAGGC
GCCAGACTCCAAGTTCGCCCTCGAGGAACTCAAGGCTACGGTCATTCCAC
GGGTGCAGGCTCGCCACACTGACCTCACCCATCTCAGCCTATCCAAGTTC
GAGTTCCATGCTGAAGCAATCTTCCTGCTCCCCTCTGTACCCATCGATGA
TCCAAAAAATGTTCCAAGTCATGACACGGTGCGCAGGACGAAGAGGGAGA
TCGCGCATATGCACGACTACCATGACTTTACGTTGCATCTTGCACTGGCC
GCCCAAGACGGGAAGGAAGTCGTGGCGAAGGGATGGGGGCAGCGACACCC
GCTGGCAGGCCCTGGCGTTCCTGGTCCACCTACGGAGTGGACGTTTATTT
ATGCGCCACGTAACGAAGAGGAACTGGCAGTGGTGGAAATGATTATCGAG
GCATCAATAGGCTATATGACCAATGACCCTGCTGGAACAGTTATCGTA
Mycena citricolor_amino acid sequence
Genbank ID: BBH43506
SEQ ID NO: 11
MAYQLTWIQTLVLGALVAMAVAFPFIKKDYETFLKGGPSYAPQNVRGYII
VLVLALFRQEQLGLEIYDRMPEKRRWLANLPQREGPRPKTTSHIIQRQLS
QHTDPAFGAAYLKDTVIPRVQARHAANTHIARSTFEFHAAAIFLNADVPL
PEGLPASETVRRTKGEIAHMHDYHDFTLHLALAAADGKEVVGKGWGQRHP
LAGPGVPGPPNEWTFVYAPRNEEEMGVVEQIVEAAIGYMSNVPALE
Mycena citricolor_nucleotide sequence
Genbank ID: LC435376
SEQ ID NO: 12
ATGGCTTATCAGCTCACTTGGATTCAGACTCTCGTGCTGGGTGCCCTTGT
GGCAATGGCAGTAGCGTTCCCCTTCATCAAGAAAGACTACGAGACGTTCC
TGAAGGGCGGCCCCTCCTATGCGCCCCAAAACGTTCGCGGATACATCATC
GTGCTCGTGCTCGCGCTCTTCCGCCAAGAGCAGCTCGGGCTGGAGATCTA
CGACCGCATGCCCGAGAAACGTCGCTGGCTCGCGAATCTCCCTCAGCGCG
AGGGCCCCCGCCCCAAGACCACAAGTCACATCATCCAGCGGCAGCTCAGC
CAGCACACGGACCCCGCATTCGGCGCCGCGTACCTCAAAGACACCGTCAT
TCCGCGCGTCCAGGCGCGGCACGCAGCCAACACGCACATCGCGCGCTCGA
CGTTCGAGTTCCACGCCGCCGCGATCTTCCTGAACGCGGACGTGCCGCTG
CCCGAGGGCCTGCCCGCAAGCGAGACGGTGCGGCGGACCAAGGGCGAGAT
CGCGCACATGCACGACTACCACGACTTCACGCTGCACCTCGCGCTCGCGG
CGGCGGATGGGAAGGAGGTGGTCGGCAAGGGCTGGGGGCAGCGCCATCCG
CTGGCGGGACCCGGTGTGCCGGGTCCGCCGAACGAGTGGACCTTTGTGTA
TGCGCCGAGGAATGAAGAGGAGATGGGCGTGGTCGAGCAGATCGTAGAGG
CGGCGATTGGGTACATGTCGAACGTGCCTGCGCTGGAA
Neonothopanus gardneri_amino acid sequence
Genbank ID: BBH43508
SEQ ID NO: 13
MNLPSFVQRLSTASSRSIAITCVVVLASAIAFPFIRRDYQTFLEVGPSYA
PQNFRGYIIVCVLSLFRQEQKGLEIYDRLPEKRRWLSDLPFRDGPRPSIT
SHIIQRQRTQLVDPDFATQELIGKVIPRVQARHTDKTFLSTSKFEFHAKA
IFLLPSIPINDPLNVPSHDTVRRTKREIAHMHDYHDCTLHIALAAQDGKE
VLKKGWGQRHPLAGPGVPGPPTEWTFLYAPRNEEEVRVVEMIIEAAIGYM
TNDPAGKVVEATGK
Neonothopanus gardneri_nucleotide sequence
Genbank ID: LC435378
SEQ ID NO: 14
ATGAATCTTCCGTCTTTCGTCCAACGTCTCTCCACAGCAAGCAGTCGCAG
TATAGCGATTACTTGCGTAGTTGTCCTTGCCTCTGCAATCGCCTTTCCCT
TCATCCGCAGAGACTACCAGACCTTCCTGGAAGTGGGACCCTCGTACGCC
CCGCAGAACTTTAGAGGATACATCATCGTCTGTGTCCTCTCGTTGTTCCG
CCAAGAACAAAAAGGACTCGAAATCTACGATCGGCTCCCAGAGAAACGAA
GGTGGTTGTCCGACCTTCCCTTTCGTGACGGGCCCAGACCCAGCATCACA
AGCCATATCATTCAACGACAGCGTACCCAACTAGTTGATCCGGACTTCGC
TACCCAGGAGCTCATAGGCAAAGTCATCCCTCGTGTGCAAGCACGACACA
CCGACAAAACATTCCTCAGCACCTCCAAATTCGAATTTCACGCAAAAGCC
ATATTCCTCCTGCCTTCCATCCCAATCAACGACCCTCTGAACGTTCCAAG
CCACGACACTGTCCGACGAACGAAGCGCGAGATCGCGCATATGCATGATT
ATCATGATTGCACTCTTCACATCGCTCTCGCTGCTCAGGACGGAAAGGAG
GTTTTGAAGAAGGGATGGGGGCAACGACACCCACTCGCTGGACCTGGAGT
GCCCGGCCCACCGACGGAGTGGACGTTTCTCTATGCGCCTCGAAACGAAG
AAGAGGTTCGAGTTGTGGAGATGATTATTGAGGCTGCCATAGGTTACATG
ACGAATGATCCGGCAGGAAAAGTTGTAGAAGCCACTGGAAAG
Omphalotus olearius_amino acid sequence
Genbank ID: BBH43510
SEQ ID NO: 15
MLPAFIYKPRLVITCVFVLASALAFPFIRKDYQTFLEVGPSYAPQNLQGY
IIVCVLSLFRQEQKDVAIYDRLPEKRRWLGDLPFREGPRPSITSHIIQRQ
RTQLADAEFATKELIGKIIPRVQARHTNTTFLSTSKFEFHAQAIFLLPSI
PINDPQNIPSHDTVRRTKREIAHMHDYHDCTLHLALAAQDGKEVLEKGWG
QRHPLAGPGVPGPPTEWTFLYAPRSEEEVRVVEMIVEASVVYMTNDPADK
IVEATVQGTEE
Omphalotus olearius_nucleotide sequence
Genbank ID: LC435380
SEQ ID NO: 16
ATGCTCCCAGCTTTCATCTACAAACCAAGGCTAGTGATCACTTGTGTATT
CGTTCTGGCCTCCGCACTCGCATTTCCCTTCATACGCAAAGATTACCAGA
CTTTCCTGGAGGTGGGACCCTCGTACGCCCCGCAGAACCTCCAAGGATAC
ATCATCGTCTGTGTACTCTCTCTGTTCCGGCAAGAACAGAAAGACGTAGC
GATTTATGATCGCCTTCCTGAGAAAAGGAGGTGGTTAGGAGACCTCCCGT
TTCGCGAGGGGCCAAGACCGAGTATCACTAGCCATATCATCCAGCGACAG
CGCACCCAATTGGCTGACGCCGAGTTCGCTACCAAAGAGCTGATAGGCAA
AATCATCCCTCGCGTCCAAGCCCGACACACCAACACAACATTCCTCAGCA
CATCTAAATTCGAATTCCACGCCCAGGCCATCTTCCTTTTGCCCTCTATC
CCAATCAACGACCCTCAAAACATTCCAAGCCACGATACCGTTCGTCGCAC
GAAACGCGAGATCGCGCATATGCATGATTATCACGACTGTACGTTGCATC
TCGCACTTGCTGCTCAAGATGGGAAGGAGGTTTTAGAGAAAGGATGGGGT
CAGCGACATCCTCTTGCTGGACCTGGTGTTCCTGGCCCGCCGACGGAGTG
GACGTTTCTTTATGCACCGCGCAGCGAAGAGGAGGTTCGGGTTGTGGAGA
TGATTGTTGAGGCATCAGTTGTGTATATGACGAATGATCCTGCGGATAAA
ATCGTAGAAGCTACTGTGCAGGGTACTGAAGAA
Panellus stipticus_amino acid sequence
Genbank ID: BBH43511
SEQ ID NO: 17
MNINLKALIGVCAVLITAAVFPFVRKDYHTFLEGGPSYAPQNLQGYIIVL
VLSLFRGEETGLEIYDRLPEKRRWLEELPVREGPRPKTTSHIIQRQLNQH
VDPDFGMNSLKGSVIRRLQSRHQDITQLALSKFEFHAEAIFLRPDIAIND
PKHVPSHDTVRRTKREIAHMHDYHDYTCHLALAAQDGKQVIAKGWGQRHP
LAGPGMPGPPTEWTFLYAPRNEAEVQVLETIIEASIGYMSNAPALGGSE
Panellus stipticus_nucleotide sequence
Genbank ID: LC435381
SEQ ID NO: 18
ATGAACATCAACCTGAAAGCTCTGATCGGAGTCTGTGCCGTGCTCATCAC
CGCTGCAGTGTTCCCCTTCGTTCGTAAAGACTATCACACCTTTCTTGAAG
GTGGACCATCCTACGCGCCGCAGAATTTGCAAGGCTATATCATCGTGTTG
GTGCTCTCACTCTTTCGAGGGGAGGAGACGGGATTGGAAATATACGACCG
CTTGCCCGAAAAACGCCGCTGGCTCGAGGAGCTGCCTGTTCGCGAAGGCC
CGCGCCCAAAGACAACCAGCCACATCATTCAGAGACAATTGAATCAGCAC
GTTGACCCGGACTTCGGAATGAACTCTTTGAAAGGCTCCGTCATCCGGCG
CCTTCAATCCCGCCACCAGGACATAACTCAACTCGCACTCTCGAAATTCG
AATTCCACGCCGAGGCCATATTTCTGCGCCCCGATATCGCGATCAACGAT
CCCAAACACGTCCCGAGCCACGACACGGTGCGCCGCACAAAGCGCGAGAT
AGCTCACATGCACGACTACCATGATTACACGTGTCATTTGGCGCTCGCAG
CGCAGGATGGGAAGCAAGTGATTGCAAAAGGGTGGGGCCAGAGACATCCG
CTCGCGGGACCGGGCATGCCGGGGCCGCCGACGGAGTGGACATTTTTGTA
TGCGCCGAGGAATGAGGCGGAGGTTCAAGTGTTGGAGACGATTATCGAAG
CGTCAATCGGGTACATGTCGAACGCACCAGCCTTGGGTGGGAGCGAG
SEQ ID NO: 19:
FPXXXXDYXTFLXXGPSYAPQNXXGYXIVXVLXLFRXEXXXXXIYXXXPE
KRXWLXXLPXRXGXRPXXTSHIIQRQXXQXXDXXFXXXXLXXXXIXRXQX
RHXXXTXXXXSXFEFHAXAIFXXXXXXXXXPXXXPXXXTVRRTKXEIAHM
HDYHDXTXHLALAAXDXKXVXXKGWGQRHPLAGPGXPGPPXEWTFXYAPR
XEXEXXVXEXIXEAXXXYMXN
<222> (5) . . . (5)
<223> Xaa is Arg or Lys.
<222> (129) . . . (130)
<223> Xaa can be any naturally occurring amino
acid or none. Xaa other than the above can be any
naturally occurring amino acid.
SEQ ID NO: 29:
FPXXXXDYXTFLXXGPSYAPQNXXGYXIVXVLXLFRXEXXXXXIYXXXPE
KRXWLXXLPXRXGXRPXXTSHIIQRQXXQXXDXXFXXXXLXXXXIXRXQX
RHXXXTXXXXSXFEFHAXAIFXXXXXXXXXPXXXPXXXTVRRTKXEIAHM
HDYHDXTXHLALAAXDXKXVXXKGWGQRHPLAGPGXPGPPXEWTFXYAPR
XEXEXXVXEXIXEAXXXYMXN
<222> (3) . . . (3)
<223> Xaa is Phe, Ile or Tyr.
<222> (4) . . . (4)
<223> Xaa is Ile or Val.
<222> (5) . . . (6)
<223> Xaa is Arg or Lys.
<222> (9) . . . (9)
<223> Xaa is Gln, Glu or His.
<222> (13) . . . (13)
<223> Xaa can be any naturally occurring amino
acid
<222> (14) . . . (14)
<223> Xaa is Gly or Val.
<222> (23) . . . (23)
<223> Xaa can be any naturally occurring amino
acid
<222> (24) . . . (24)
<223> Xaa is Arg, Lys or Gln.
<222> (27) . . . (27)
<223> Xaa is Ile Phe or Leu.
<222> (30) . . . (30)
<223> Xaa is Leu or Cys.
<222> (33) . . . (33)
<223> Xaa is Ser or Ala.
<222> (37) . . . (37)
<223> Xaa is Gly or Gln.
<222> (39) . . . (39)
<223> Xaa is Glu or Gln.
<222> (40) . . . (40)
<223> Xaa is Lys, Leu or Thr.
<222>(41) . . . (41)
<223> Xaa is Gly or Asp.
<222> (42) . . . (42)
<223> Xaa is Leu or Val.
<222> (43) . . . (43)
<223> Xaa is Ala or Glu.
<222> (46) . . . (46)
<223> Xaa is Glu or Asp.
<222> (47) . . . (47)
<223> Xaa is Pro or Arg.
<222> (48) . . . (48)
<223> Xaa is Leu or Met.
<222>(53) . . . (53)
<223> Xaa is Thr or Arg.
<222> (56) . . . (56)
<223> Xaa can be any naturally occurring amino
acid
<222> (57) . . . (57)
<223> Xaa is Glu, Asp or Asn.
<222> (60) . . . (60)
<223> Xaa can be any naturally occurring amino
acid
<222> (62) . . . (62)
<223> Xaa can be any naturally occurring amino
acid
<222> (64) . . . (64)
<223> Xaa is Asp, Thr or Pro.
<222> (67) . . . (67)
<223> Xaa can be any naturally occurring amino
acid
<222> (68) . . . (68)
<223> Xaa is Thr or Ile.
<222> (77) . . . (77)
<223> Xaa is Leu or Arg.
<222> (78) . . . (78)
<223> Xaa can be any naturally occurring amino
acid
<222> (80) . . . (81)
<223> Xaa can be any naturally occurring amino
acid
<222> (83) . . . (83)
<223> Xaa is Pro, Gln, Leu, Ser or Ala.
<222> (84) . . . (84)
<223> Xaa can be any naturally occurring amino
acid
<222> (86) . . . (86)
<223> Xaa is Val, Ala or Gly.
<222> (87) . . . (88)
<223> Xaa can be any naturally occurring amino
acid
<222> (89) . . . (89)
<223> Xaa is Ala, Glu, Tyr or Ser.
<222> (91) . . . (91)
<223> Xaa is Lys, or Ile.
<222> (92) . . . (92)
<223> Xaa can be any naturally occurring amino
acid
<222> (93) . . . (93)
<223> Xaa is Thr, Lys or Ser.
<222> (94) . . . (94)
<223> Xaa is Val or Ile.
<222> (96) . . . (96)
<223> Xaa is Pro or Arg.
<222> (98) . . . (98)
<223> Xaa is Val or Leu.
<222> (100) . . . (100)
<223> Xaa is Ala or Ser.
<222> (103) . . . (103)
<223> Xaa is Thr, Ala or Gln.
<222> (104) . . . (104)
<223> Xaa is Asp, Ala or Asn.
<222> (105) . . . (105)
<223> Xaa can be any naturally occurring amino
acid
<222> (107) . . . (107)
<223> Xaa is His, Phe or Gln.
<222> (108) . . . (108)
<223> Xaa is Leu or Ile.
<222> (109) . . . (109)
<223> Xaa is Ala or Ser.
<222> (110) . . . (110)
<223> Xaa is Leu, Thr or Arg.
<222> (112) . . . (112)
<223> Xaa is Lys or Thr.
<222> (118) . . . (118)
<223> Xaa is Glu, Lys, Ala or Gln.
<222> (122) . . . (122)
<223> Xaa is Val or Leu.
<222> (123) . . . (123)
<223> Xaa is Arg, Leu, or Asn.
<222> (124) . . . (124)
<223> Xaa is Pro or Ala.
<222> (125) . . . (125)
<223> Xaa is Glu, Ser or Asp.
<222> (126) . . . (126)
<223> Xaa is Ile or Val.
<222> (127) . . . (127)
<223> Xaa is Ala or Pro.
<222> (128) . . . (128)
<223> Xaa is Ile or Leu.
<222> (129) . . . (129)
<223> Xaa can be any naturally occurring amino
acid or none.
<222> (130) . . . (130)
<223> Xaa is Asp or none.
<222> (132) . . . (132)
<223> Xaa is Lys, Leu, Glu, or Gln.
<222> (133) . . . (133)
<223> Xaa is His, Asn or Gly.
<222> (134) . . . (134)
<223> Xaa can be any naturally occurring amino
acid
<222> (136) . . . (136)
<223> Xaa is Ser or Ala.
<222> (137) . . . (137)
<223> Xaa is His or Ser.
<222> (138) . . . (138)
<223> Xaa is Asp or Glu.
<222> (145) . . . (145)
<223> Xaa is Arg or Gly.
<222> (156) . . . (156)
<223> Xaa can be any naturally occurring amino
acid
<222> (158) . . . (158)
<223> Xaa is Leu or Cys.
<222> (160) . . . (160)
<223> Xaa is Leu or Ile.
<222> (165) . . . (165)
<223> Xaa is Gln or Ala.
<222> (167) . . . (167)
<223> Xaa is Ala or Gly.
<222> (169) . . . (169)
<223> Xaa is Gln or Glu.
<222> (171) . . . (172)
<223> Xaa can be any naturally occurring amino
acid
<222> (186) . . . (186)
<223> Xaa can be any naturally occurring amino
acid
<222> (191) . . . (191)
<223> Xaa can be any naturally occurring amino
acid
<222>(196) . . . (196)
<223> Xaa can be any naturally occurring amino
acid
<222> (201) . . . (201)
<223> Xaa is Thr, Asn or Ser.
<222> (203) . . . (203)
<223> Xaa is Glu or Ala.
<222> (205) . . . (206)
<223> Xaa can be any naturally occurring amino
acid
<222> (208) . . . (208)
<223> Xaa is Val or Leu.
<222> (210) . . . (210)
<223> Xaa is Thr, Met or Gln.
<222> (212) . . . (212)
<223> Xaa can be any naturally occurring amino
acid
<222> (215) . . . (215)
<223> Xaa is Ser or Ala.
<222> (216) . . . (216)
<223> Xaa is Ile or Val.
<222> (217) . . . (217)
<223> Xaa is Ala, Gly or Val.
<222> (220) . . . (220)
<223> Xaa is Thr or Ser.

1: A modified luciferase derived from a luminescent mushroom or a functional fragment thereof, comprising a mutation introduced into arginine or lysine corresponding to position 26 of amino acid sequence shown in SEQ ID NO: 1 and/or a mutation introduced into valine corresponding to position 161 of amino acid sequence shown in SEQ ID NO: 1, and having enhanced luminescence compared with a luciferase before the mutation is introduced.

2: The modified luciferase or functional fragment thereof according to claim 1,
wherein the modified luciferase is a variant obtained by modifying a luciferase from a luminescent mushroom having an amino acid sequence as set forth in SEQ ID NO: 19:
FPXXXXDYXTFLXXGPSYAPQNXXGYXIVXVLXLFRXEXXXXXIYXXXPE
KRXWLXXLPXRXGXRPXXTSHIIQRQXXQXXDXXFXXXXLXXXXIXRXQX
RHXXXTXXXXSXFEFHAXAIFXXXXXXXXXPXXXPXXXTVRRTKXEIAHM
HDYHDXTXHLALAAXDXKXVXXKGWGQRHPLAGPGXPGPPXEWTFXYAPR
XEXEXXVXEXIXEAXXXYMXN.

3: An isolated nucleic acid encoding the modified luciferase or functional fragment thereof according to claim 1.

4: An isolated nucleic acid encoding a modified luciferase or a functional fragment thereof having any amino acid sequence selected from the group consisting of (a) to (c) below, and a mutation introduced into arginine or lysine corresponding to position 26 of amino acid sequence shown in SEQ ID NO: 1, and/or a mutation introduced into valine corresponding to position 161 of amino acid sequence shown in SEQ ID NO: 1 in the amino acid sequence selected from the group consisting of (a) to (c) below:
(a) an amino acid sequence of a luciferase derived from a luminescent mushroom, which is set forth in SEQ ID NO: 1, 3, 5, 7, 9, 11, 13, 15, or 17;
(b) an amino acid sequence that is at least 80% identical to the amino acid sequence set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine; and
(c) an amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine.

5: The isolated nucleic acid according to claim 4, wherein the modified luciferase has enhanced luminescence compared with a luciferase before the mutation is introduced.

6: An expression cassette under the control of a regulatory element required for the expression of a nucleic acid in a host cell, comprising the nucleic acid according to claim 3,
wherein the expression cassette is integrated into a genome of a cell or introduced into the cell in the form of an extrachromosomal element, and
the expression cassette is capable of causing the expression of a luciferase encoded by the nucleic acid.

7: A cell producing a luciferase encoded by the nucleic acid according to claim 3, comprising:
an expression cassette under the control of a regulatory element required for the expression of a nucleic acid in a host cell, the expression cassette containing the nucleic acid,
wherein the expression cassette is integrated into a genome of the cell or introduced into the cell in the form of an extrachromosomal element, and
the expression cassette in the form of the extrachromosomal element or an element integrated into the genome of the cell is capable of causing the expression of the luciferase encoded by the nucleic acid.

8: A modified luciferase or a functional fragment thereof having any amino acid sequence selected from the group consisting of (a) to (c) below, and a mutation introduced into arginine or lysine corresponding to position 26 of amino acid sequence shown in SEQ ID NO:1, and/or a mutation introduced into valine corresponding to position 161 of amino acid sequence shown in SEQ ID NO: 1, in the amino acid sequence selected from the group consisting of (a) to (c) below:
(a) an amino acid sequence of a luciferase derived from a luminescent mushroom, which is set forth in SEQ ID NO: 1, 3, 5, 7, 9, 11, 13, 15, or 17;
(b) an amino acid sequence that is at least 80% identical to the amino acid sequence set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine; and
(c) an amino acid sequence that has an amino acid sequence including one to several amino acid substitutions, deletions, insertions, and/or additions relative to the amino acid sequence as set forth in SEQ ID NO: 1, 3, 5, 7, 9, 11, 13, 15, or 17, wherein an amino acid corresponding to position 26 in the amino acid sequence shown in SEQ ID NO: 1 is arginine or lysine, and an amino acid corresponding to position 161 in the amino acid sequence shown in SEQ ID NO: 1 is valine.

9: The modified luciferase or functional fragment thereof according to claim 8, wherein the modified luciferase has enhanced luminescence compared with a luciferase before the mutation is introduced.

10: A fusion protein obtained by fusing the modified luciferase or functional fragment thereof according to claim 1 to another protein.

11: A fusion protein, wherein the modified luciferase or functional fragment thereof according to claim 1 is linked to a fluorescent protein via a linker sequence in such a manner that enables resonance energy transfer from the modified luciferase or functional fragment thereof to the fluorescent protein.

12: An isolated nucleic acid encoding the fusion protein according to claim 10.

13. (canceled)

14: A transgenic organism comprising the nucleic acid according to claim 3.

15. A kit comprising at least one selected from the group consisting of (1) to (2) below and a luminescent substrate for a modified luciferase:
(1) the nucleic acid according to claim 3; and
(2) an expression cassette containing the isolated nucleic acid;
wherein the luminescent substrate for modified luciferase is a luminescent substrate for the modified luciferase derived from a luminescent mushroom or a functional fragment thereof, comprising a mutation introduced into arginine or lysine corresponding to position 26 of amino acid sequence shown in SEQ ID NO: land/or a mutation introduced into valine corresponding to position 161 of amino acid sequence shown in SEQ ID NO: 1, and having enhanced luminescence compared with a luciferase before the mutation is introduced.

16. A kit comprising at least one selected from the group consisting of (1) to (2) below and a luminescent substrate for a modified luciferase according claim 1:
(1) the modified luciferase or functional fragment thereof according to claim 1; and
(2) a fusion protein obtained by fusing the modified luciferase or functional fragment thereof to another protein.


SEQUENCE LISTING (29 sequences)

SEQ ID NO: 1 - protein, 251 aa, Mycena chlorophos
MVQLTRTSGFIAAAAIVAAIAFPFIRRDYQTFLRGGPSYAPQNIRGYIIVLVLSLFRGEEKGLAIYEPLPEKRTWLPELPRRAGDRPKTTSHIIQRQLDQYPDPDFVLKALKATVIPRVQARHTDKTHLALSKFEFHAEAIFVRPEIAIDDPKHIPSHDTVRRTKREIAHMHDYHDCTLHLALAAQDAKQVLQKGWGQRHPLAGPGMPGPPTEWTFLYAPRTEEEVKVVETIVEASIAYMTNAEKPVELVQ

SEQ ID NO: 2 - DNA, 753 bp, Mycena chlorophos
  source 1..753: mol_type = genomic DNA
atggtccaactcacccgaacctccggattcatcgccgctgcggccattgttgctgccatcgccttcccgttcattcgtcgagactaccagacgttccttcgtggtgggccgtcctatgccccacagaacatccgcggctatatcatcgtcctggttctgtccctcttccgcggcgaggagaagggtcttgcaatctacgagccccttcctgagaagcgcacatggctgccggagcttccgcggcgcgcgggagaccggcccaagacgacgagccacatcatccaacggcagctcgaccagtaccccgacccggactttgtcctcaaagccctgaaagcgacggtcatcccgcgtgtccaagcccggcacacagacaagactcacctcgcgctgtccaagttcgagttccatgctgaggccatcttcgtgcgcccggaaatcgccatcgacgacccgaagcatatccccagccacgacacggtgcgacggacgaagcgcgagattgcgcacatgcacgactatcacgactgcacgctgcatttggcgctagcggcgcaggacgcgaagcaggtgctgcagaagggctggggccagcgccatccgctggcagggcctgggatgcccgggccgcccacggagtggacgttcttgtatgccccgaggaccgaggaggaagtgaaggttgtggagaccattgtcgaggcctctatcgcgtacatgacgaacgcggagaagccggtcgagctggtgcag

SEQ ID NO: 3 - protein, 267 aa, Neonothopanus nambi
MRINISLSSLFERLSKLSSRSIAITCGVVLASAIAFPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTNDPAGKIVENAK

SEQ ID NO: 4 - DNA, 801 bp, Neonothopanus nambi
  source 1..801: mol_type = genomic DNA
atgcgcattaacattagcctctcgtctctcttcgaacgtctctccaaacttagcagtcgcagcatagcgattacatgtggagttgttctcgcctccgcaatcgcctttcccatcatccgcagagactaccagactttcctagaagtgggaccctcgtacgctccgcagaactttagaggatacatcatcgtctgtgtcctctcgctattccgccaagagcagaaagggctcgccatctatgatcgtcttcccgagaaacgcaggtggttggccgaccttccctttcgtgaaggaaccagacccagcattaccagccatatcattcagcgacagcgcactcaactggtcgatcaggagtttgccaccagggagctcatagacaaggtcatccctcgcgtgcaagcacgacacaccgacaaaacgttcctcagcacatcaaagttcgagtttcatgcgaaggccatatttctcttgccttctatcccaatcaacgaccctctgaatatccctagccacgacactgtccgccgaacgaagcgcgagattgcacatatgcatgattatcatgattgcacacttcatcttgctctcgctgcgcaggatggaaaggaggtgctgaagaaaggttggggacaacgacatcctttggctggtcctggagttcctggtccaccaacggaatggacttttctttatgcgcctcgcaacgaagaagaggctcgagtagtggagatgatcgttgaggcttccatagggtatatgacgaacgatcctgcaggaaagattgtagaaaacgccaag

SEQ ID NO: 5 - protein, 266 aa, Armillaria gallica
MSFIDSMKLDLVGHLFGIRNRGLAAACCALAVASTIAFPYIRRDYQTFLSGGPSYAPQNIRGYFIVCVLALFRQEQKGLAIYDRLPEKRRWLPDLPPRNGPRPITTSHIIQRQRNQAPDPKFALEELKATVIPRVQARHTDLTHLSLSKFEFHAEAIFLLPSVPIDDPKNVPSHDTVRRTKREIAHMHDYHDFTLHLALAAQDGKEVVSKGWGQRHPLAGPGVPGPPTEWTFIYAPRNEEELAVVEMIIEASIGYMTNDPAGVVIA

SEQ ID NO: 6 - DNA, 798 bp, Armillaria gallica
  source 1..798: mol_type = genomic DNA
atgtccttcatcgacagcatgaaacttgacctcgtcggacacctctttggcatcaggaatcgcggcttagccgccgcttgttgtgctctagcagtcgcctctactatcgccttcccttacattcgtagggactaccagacatttttatctggcggtccctcttacgctccccagaatatcagaggatatttcatcgtctgcgttctggccttgttccgtcaggagcaaaagggccttgcgatatatgatcgccttcccgagaagcgcaggtggctgcctgacttgcctcctcgcaatggcccgcggccgatcacgaccagccatataatccaaagacagcgcaaccaggcgccggaccccaagttcgccctcgaggaactcaaggccacggttattccacgggtgcaggctcgccatactgacctcacccatctcagcctatccaaattcgagttccatgctgaagcaattttcctgctcccctctgtacccatcgatgatccaaaaaatgttccaagtcacgacacggtgcgcaggacgaaaagggagatcgcgcatatgcacgactaccatgacttcacgctgcatcttgcactggccgcccaagacgggaaggaagtcgtgtcgaagggatgggggcagcgacaccccctagcaggccctggcgttcctggtccacctacggagtggacatttatttatgcgccacgtaacgaagaggaactggcagtggtggaaatgattatcgaggcatcaataggctatatgaccaatgaccctgctggagtagttatcgca

SEQ ID NO: 7 - protein, 266 aa, Armillaria mellea
MSFFDSVKLDLVGRLFGIRNRGLAVTCCAVAVASIIAFPYIRRDYQTFLSGGPSYAPQNIRGYLIVCVLALFRQEQKGLAIYDRLPEKRRWLPDLPPRDGPRPITTSHIIQRQRNQAPDLKFALEELKATVIPRVQARHTDLTHLSLSKFEFHAEAIFLLPSVPIDDPKNVPSHDTVRRTKREIAHMHDYHDYTLHLALAAQDGKEVVSKGWGQRHPLAGPGVPGPPTEWTFIYAPRNEEELAVVEMIIEASIGYMTNDPAGKTIA

SEQ ID NO: 8 - DNA, 798 bp, Armillaria mellea
  source 1..798: mol_type = genomic DNA
atgtccttcttcgacagcgtgaaacttgacctcgtcggacgcctctttggcatcaggaatcgcggcttagctgttacttgttgtgctgtggcagtcgcctctatcatcgcgttcccttacattcgtagggactaccagacatttttatctgggggtccctcctacgctccccagaacatcagaggatacctcattgtctgcgtcctggccttgttccgtcaggagcaaaaaggccttgcgatatacgaccgccttcccgagaagcgcaggtggctacctgacttgcctcctcgcgatggcccacggcccatcacgaccagccatataatccaaagacagcgcaaccaggcgccggacctcaagttcgccctcgaggaactcaaggccacggtcattccacgggtgcaggctcgccacactgacctcacccatctcagcctatccaagttcgagttccatgctgaagcaatcttcctgctcccctctgtacccatcgatgatccaaagaatgtgccaagtcacgacacggtgcgcaggacgaagagggaaattgcgcatatgcacgactaccatgactacacgctgcatcttgcgttggccgcccaagacgggaaggaagtcgtatcaaagggatgggggcagcgacacccgctggcaggccctggcgttcctggtccaccgacggagtggacgtttatttatgcgccacgtaacgaagaggagctggcagtggtggaaatgattatcgaggcatcgataggctatatgaccaatgaccctgcaggaaaaactatcgca

SEQ ID NO: 9 - protein, 266 aa, Armillaria ostoyae
MSFIDSMKLDFVGHLFGIRNRGLATACCAVAVASAIAFPYIRRDYQTFLSGGPSYAPQNIKGYLIVCVLALFRQEQKGLAIYDRLPEKRRWLPDLPPRNGPRPITTSHIIQRQRNQAPDSKFALEELKATVIPRVQARHTDLTHLSLSKFEFHAEAIFLLPSVPIDDPKNVPSHDTVRRTKREIAHMHDYHDFTLHLALAAQDGKEVVAKGWGQRHPLAGPGVPGPPTEWTFIYAPRNEEELAVVEMIIEASIGYMTNDPAGTVIV

SEQ ID NO: 10 - DNA, 798 bp, Armillaria ostoyae
  source 1..798: mol_type = genomic DNA
atgtccttcatcgacagcatgaaacttgacttcgtcggacacctctttggcatcaggaatcgcggcttagccaccgcttgttgtgctgtggcagtcgcttctgccatcgccttcccttacattcgtagggactaccagacattcttatctggcggtccctcttacgctccccagaacatcaaaggatatctcatcgtctgcgtcctggccttgttccgtcaggagcaaaagggccttgcgatatatgaccgccttcccgagaagcgcaggtggctacctgacttgcctcctcgcaatggcccgcggcccatcacgaccagccatataatccaaagacagcgcaaccaggcgccagactccaagttcgccctcgaggaactcaaggctacggtcattccacgggtgcaggctcgccacactgacctcacccatctcagcctatccaagttcgagttccatgctgaagcaatcttcctgctcccctctgtacccatcgatgatccaaaaaatgttccaagtcatgacacggtgcgcaggacgaagagggagatcgcgcatatgcacgactaccatgactttacgttgcatcttgcactggccgcccaagacgggaaggaagtcgtggcgaagggatgggggcagcgacacccgctggcaggccctggcgttcctggtccacctacggagtggacgtttatttatgcgccacgtaacgaagaggaactggcagtggtggaaatgattatcgaggcatcaataggctatatgaccaatgaccctgctggaacagttatcgta

SEQ ID NO: 11 - protein, 246 aa, Mycena citricolor
MAYQLTWIQTLVLGALVAMAVAFPFIKKDYETFLKGGPSYAPQNVRGYIIVLVLALFRQEQLGLEIYDRMPEKRRWLANLPQREGPRPKTTSHIIQRQLSQHTDPAFGAAYLKDTVIPRVQARHAANTHIARSTFEFHAAAIFLNADVPLPEGLPASETVRRTKGEIAHMHDYHDFTLHLALAAADGKEVVGKGWGQRHPLAGPGVPGPPNEWTFVYAPRNEEEMGVVEQIVEAAIGYMSNVPALE

SEQ ID NO: 12 - DNA, 738 bp, Mycena citricolor
  source 1..738: mol_type = genomic DNA
atggcttatcagctcacttggattcagactctcgtgctgggtgcccttgtggcaatggcagtagcgttccccttcatcaagaaagactacgagacgttcctgaagggcggcccctcctatgcgccccaaaacgttcgcggatacatcatcgtgctcgtgctcgcgctcttccgccaagagcagctcgggctggagatctacgaccgcatgcccgagaaacgtcgctggctcgcgaatctccctcagcgcgagggcccccgccccaagaccacaagtcacatcatccagcggcagctcagccagcacacggaccccgcattcggcgccgcgtacctcaaagacaccgtcattccgcgcgtccaggcgcggcacgcagccaacacgcacatcgcgcgctcgacgttcgagttccacgccgccgcgatcttcctgaacgcggacgtgccgctgcccgagggcctgcccgcaagcgagacggtgcggcggaccaagggcgagatcgcgcacatgcacgactaccacgacttcacgctgcacctcgcgctcgcggcggcggatgggaaggaggtggtcggcaagggctgggggcagcgccatccgctggcgggacccggtgtgccgggtccgccgaacgagtggacctttgtgtatgcgccgaggaatgaagaggagatgggcgtggtcgagcagatcgtagaggcggcgattgggtacatgtcgaacgtgcctgcgctggaa

SEQ ID NO: 13 - protein, 264 aa, Neonothopanus gardneri
MNLPSFVQRLSTASSRSIAITCVVVLASAIAFPFIRRDYQTFLEVGPSYAPQNFRGYIIVCVLSLFRQEQKGLEIYDRLPEKRRWLSDLPFRDGPRPSITSHIIQRQRTQLVDPDFATQELIGKVIPRVQARHTDKTFLSTSKFEFHAKAIFLLPSIPINDPLNVPSHDTVRRTKREIAHMHDYHDCTLHIALAAQDGKEVLKKGWGQRHPLAGPGVPGPPTEWTFLYAPRNEEEVRVVEMIIEAAIGYMTNDPAGKVVEATGK

SEQ ID NO: 14 - DNA, 792 bp, Neonothopanus gardneri
  source 1..792: mol_type = genomic DNA
atgaatcttccgtctttcgtccaacgtctctccacagcaagcagtcgcagtatagcgattacttgcgtagttgtccttgcctctgcaatcgcctttcccttcatccgcagagactaccagaccttcctggaagtgggaccctcgtacgccccgcagaactttagaggatacatcatcgtctgtgtcctctcgttgttccgccaagaacaaaaaggactcgaaatctacgatcggctcccagagaaacgaaggtggttgtccgaccttccctttcgtgacgggcccagacccagcatcacaagccatatcattcaacgacagcgtacccaactagttgatccggacttcgctacccaggagctcataggcaaagtcatccctcgtgtgcaagcacgacacaccgacaaaacattcctcagcacctccaaattcgaatttcacgcaaaagccatattcctcctgccttccatcccaatcaacgaccctctgaacgttccaagccacgacactgtccgacgaacgaagcgcgagatcgcgcatatgcatgattatcatgattgcactcttcacatcgctctcgctgctcaggacggaaaggaggttttgaagaagggatgggggcaacgacacccactcgctggacctggagtgcccggcccaccgacggagtggacgtttctctatgcgcctcgaaacgaagaagaggttcgagttgtggagatgattattgaggctgccataggttacatgacgaatgatccggcaggaaaagttgtagaagccactggaaag

SEQ ID NO: 15 - protein, 261 aa, Omphalotus olearius
MLPAFIYKPRLVITCVFVLASALAFPFIRKDYQTFLEVGPSYAPQNLQGYIIVCVLSLFRQEQKDVAIYDRLPEKRRWLGDLPFREGPRPSITSHIIQRQRTQLADAEFATKELIGKIIPRVQARHTNTTFLSTSKFEFHAQAIFLLPSIPINDPQNIPSHDTVRRTKREIAHMHDYHDCTLHLALAAQDGKEVLEKGWGQRHPLAGPGVPGPPTEWTFLYAPRSEEEVRVVEMIVEASVVYMTNDPADKIVEATVQGTEE

SEQ ID NO: 16 - DNA, 783 bp, Omphalotus olearius
  source 1..783: mol_type = genomic DNA
atgctcccagctttcatctacaaaccaaggctagtgatcacttgtgtattcgttctggcctccgcactcgcatttcccttcatacgcaaagattaccagactttcctggaggtgggaccctcgtacgccccgcagaacctccaaggatacatcatcgtctgtgtactctctctgttccggcaagaacagaaagacgtagcgatttatgatcgccttcctgagaaaaggaggtggttaggagacctcccgtttcgcgaggggccaagaccgagtatcactagccatatcatccagcgacagcgcacccaattggctgacgccgagttcgctaccaaagagctgataggcaaaatcatccctcgcgtccaagcccgacacaccaacacaacattcctcagcacatctaaattcgaattccacgcccaggccatcttccttttgccctctatcccaatcaacgaccctcaaaacattccaagccacgataccgttcgtcgcacgaaacgcgagatcgcgcatatgcatgattatcacgactgtacgttgcatctcgcacttgctgctcaagatgggaaggaggttttagagaaaggatggggtcagcgacatcctcttgctggacctggtgttcctggcccgccgacggagtggacgtttctttatgcaccgcgcagcgaagaggaggttcgggttgtggagatgattgttgaggcatcagttgtgtatatgacgaatgatcctgcggataaaatcgtagaagctactgtgcagggtactgaagaa

SEQ ID NO: 17 - protein, 249 aa, Panellus stipticus
MNINLKALIGVCAVLITAAVFPFVRKDYHTFLEGGPSYAPQNLQGYIIVLVLSLFRGEETGLEIYDRLPEKRRWLEELPVREGPRPKTTSHIIQRQLNQHVDPDFGMNSLKGSVIRRLQSRHQDITQLALSKFEFHAEAIFLRPDIAINDPKHVPSHDTVRRTKREIAHMHDYHDYTCHLALAAQDGKQVIAKGWGQRHPLAGPGMPGPPTEWTFLYAPRNEAEVQVLETIIEASIGYMSNAPALGGSE

SEQ ID NO: 18 - DNA, 747 bp, Panellus stipticus
  source 1..747: mol_type = genomic DNA
atgaacatcaacctgaaagctctgatcggagtctgtgccgtgctcatcaccgctgcagtgttccccttcgttcgtaaagactatcacacctttcttgaaggtggaccatcctacgcgccgcagaatttgcaaggctatatcatcgtgttggtgctctcactctttcgaggggaggagacgggattggaaatatacgaccgcttgcccgaaaaacgccgctggctcgaggagctgcctgttcgcgaaggcccgcgcccaaagacaaccagccacatcattcagagacaattgaatcagcacgttgacccggacttcggaatgaactctttgaaaggctccgtcatccggcgccttcaatcccgccaccaggacataactcaactcgcactctcgaaattcgaattccacgccgaggccatatttctgcgccccgatatcgcgatcaacgatcccaaacacgtcccgagccacgacacggtgcgccgcacaaagcgcgagatagctcacatgcacgactaccatgattacacgtgtcatttggcgctcgcagcgcaggatgggaagcaagtgattgcaaaagggtggggccagagacatccgctcgcgggaccgggcatgccggggccgccgacggagtggacatttttgtatgcgccgaggaatgaggcggaggttcaagtgttggagacgattatcgaagcgtcaatcgggtacatgtcgaacgcaccagccttgggtgggagcgag

SEQ ID NO: 19 - protein, 221 aa, synthetic construct
  REGION 1..221: note = Consensus sequence
  REGION 3..4: note = misc_feature - Xaa can be any naturally occurring amino acid
  SITE 5: note = MISC_FEATURE - Xaa is Arg or Lys.
  SITE 6: note = misc_feature - Xaa can be any naturally occurring amino acid
  SITE 9: note = misc_feature - Xaa can be any naturally occurring amino acid
  REGION 13..14: note = misc_feature - Xaa can be any naturally occurring amino acid
  REGION 23..24: note = misc_feature - Xaa can be any naturally occurring amino acid
  SITE 27: note = misc_feature - Xaa can be any naturally occurring amino acid
  SITE 30: note = misc_feature - Xaa can be any naturally occurring amino acid
  SITE 33: note = misc_feature - Xaa can be any naturally occurring amino acid
  SITE 37: note = misc_feature - Xaa can be any naturally occurring amino acid
  REGION 39..43: note = misc_feature - Xaa can be any naturally occurring amino acid
  REGION 46..48: note = misc_feature - Xaa can be any naturally occurring amino acid
  SITE 53: note = misc_feature - Xaa can be any naturally occurring amino acid
  REGION 56..57: note = misc_feature - Xaa can be any naturally occurring amino acid
  SITE 60: note = misc_feature - Xaa can be any naturally occurring amino acid
  SITE 62: note = misc_feature - Xaa can be any naturally occurring amino acid
  SITE 64: note = misc_feature - Xaa can be any naturally occurring amino acid
  REGION 67..68: note = misc_feature - Xaa can be any naturally occurring amino acid
  REGION 77..78: note = misc_feature - Xaa can be any naturally occurring amino acid
  REGION 80..81: note = misc_feature - Xaa can be any naturally occurring amino acid
  REGION 83..84: note = misc_feature - Xaa can be any naturally occurring amino acid
  REGION 86..89: note = misc_feature - Xaa can be any naturally occurring amino acid
  REGION 91..94: note = misc_feature - Xaa can be any naturally occurring amino acid
  SITE 96: note = misc_feature - Xaa can be any naturally occurring amino acid
  SITE 98: note = misc_feature - Xaa can be any naturally occurring amino acid
  SITE 100: note = misc_feature - Xaa can be any naturally occurring amino acid
  REGION 103..105: note = misc_feature - Xaa can be any naturally occurring amino acid
  REGION 107..110: note = misc_feature - Xaa can be any naturally occurring amino acid
  SITE 112: note = misc_feature - Xaa can be any naturally occurring amino acid
  SITE 118: note = misc_feature - Xaa can be any naturally occurring amino acid
  REGION 122..128: note = misc_feature - Xaa can be any naturally occurring amino acid
  REGION 132..134: note = misc_feature - Xaa can be any naturally occurring amino acid
  REGION 136..138: note = misc_feature - Xaa can be any naturally occurring amino acid
  SITE 145: note = misc_feature - Xaa can be any naturally occurring amino acid
  SITE 156: note = misc_feature - Xaa can be any naturally occurring amino acid
  SITE 158: note = misc_feature - Xaa can be any naturally occurring amino acid
  SITE 165: note = misc_feature - Xaa can be any naturally occurring amino acid
  SITE 167: note = misc_feature - Xaa can be any naturally occurring amino acid
  SITE 169: note = misc_feature - Xaa can be any naturally occurring amino acid
  REGION 171..172: note = misc_feature - Xaa can be any naturally occurring amino acid
  SITE 186: note = misc_feature - Xaa can be any naturally occurring amino acid
  SITE 191: note = misc_feature - Xaa can be any naturally occurring amino acid
  SITE 196: note = misc_feature - Xaa can be any naturally occurring amino acid
  SITE 201: note = misc_feature - Xaa can be any naturally occurring amino acid
  SITE 203: note = misc_feature - Xaa can be any naturally occurring amino acid
  REGION 205..206: note = misc_feature - Xaa can be any naturally occurring amino acid
  SITE 208: note = misc_feature - Xaa can be any naturally occurring amino acid
  SITE 210: note = misc_feature - Xaa can be any naturally occurring amino acid
  SITE 212: note = misc_feature - Xaa can be any naturally occurring amino acid
  REGION 215..217: note = misc_feature - Xaa can be any naturally occurring amino acid
  SITE 220: note = misc_feature - Xaa can be any naturally occurring amino acid
  REGION 129..130: note = misc_feature - Xaa can be any naturally occurring amino acid or none.
FPXXXXDYXTFLXXGPSYAPQNXXGYXIVXVLXLFRXEXXXXXIYXXXPEKRXWLXXLPXRXGXRPXXTSHIIQRQXXQXXDXXFXXXXLXXXXIXRXQXRHXXXTXXXXSXFEFHAXAIFXXXXXXXXXPXXXPXXXTVRRTKXEIAHMHDYHDXTXHLALAAXDXKXVXXKGWGQRHPLAGPGXPGPPXEWTFXYAPRXEXEXXVXEXIXEAXXXYMXN

SEQ ID NO: 20 - protein, 221 aa, synthetic construct
  REGION 1..221: note = Amino acid sequence of the functional fragment of lusiferase from Mycena chlorophos (22 - 242)
FPFIRRDYQTFLRGGPSYAPQNIRGYIIVLVLSLFRGEEKGLAIYEPLPEKRTWLPELPRRAGDRPKTTSHIIQRQLDQYPDPDFVLKALKATVIPRVQARHTDKTHLALSKFEFHAEAIFVRPEIAIDDPKHIPSHDTVRRTKREIAHMHDYHDCTLHLALAAQDAKQVLQKGWGQRHPLAGPGMPGPPTEWTFLYAPRTEEEVKVVETIVEASIAYMTN

SEQ ID NO: 21 - protein, 221 aa, synthetic construct
  REGION 1..221: note = Amino acid sequence of the functional fragment of lusiferase from Neonothopanus nambi (36-256)
FPIIRRDYQTFLEVGPSYAPQNFRGYIIVCVLSLFRQEQKGLAIYDRLPEKRRWLADLPFREGTRPSITSHIIQRQRTQLVDQEFATRELIDKVIPRVQARHTDKTFLSTSKFEFHAKAIFLLPSIPINDPLNIPSHDTVRRTKREIAHMHDYHDCTLHLALAAQDGKEVLKKGWGQRHPLAGPGVPGPPTEWTFLYAPRNEEEARVVEMIVEASIGYMTN

SEQ ID NO: 22 - protein, 221 aa, synthetic construct
  REGION 1..221: note = Amino acid sequence of the functional fragment of lusiferase from Armillaria gallica (38-258)
FPYIRRDYQTFLSGGPSYAPQNIRGYFIVCVLALFRQEQKGLAIYDRLPEKRRWLPDLPPRNGPRPITTSHIIQRQRNQAPDPKFALEELKATVIPRVQARHTDLTHLSLSKFEFHAEAIFLLPSVPIDDPKNVPSHDTVRRTKREIAHMHDYHDFTLHLALAAQDGKEVVSKGWGQRHPLAGPGVPGPPTEWTFIYAPRNEEELAVVEMIIEASIGYMTN

SEQ ID NO: 23 - protein, 221 aa, synthetic construct
  REGION 1..221: note = Amino acid sequence of the functional fragment of lusiferase from Armillaria mellea (38-258)
FPYIRRDYQTFLSGGPSYAPQNIRGYLIVCVLALFRQEQKGLAIYDRLPEKRRWLPDLPPRDGPRPITTSHIIQRQRNQAPDLKFALEELKATVIPRVQARHTDLTHLSLSKFEFHAEAIFLLPSVPIDDPKNVPSHDTVRRTKREIAHMHDYHDYTLHLALAAQDGKEVVSKGWGQRHPLAGPGVPGPPTEWTFIYAPRNEEELAVVEMIIEASIGYMTN

SEQ ID NO: 24 - protein, 221 aa, synthetic construct
  REGION 1..221: note = Amino acid sequence of the functional fragment of lusiferase from Armillaria ostoyae (38-258)
FPYIRRDYQTFLSGGPSYAPQNIKGYLIVCVLALFRQEQKGLAIYDRLPEKRRWLPDLPPRNGPRPITTSHIIQRQRNQAPDSKFALEELKATVIPRVQARHTDLTHLSLSKFEFHAEAIFLLPSVPIDDPKNVPSHDTVRRTKREIAHMHDYHDFTLHLALAAQDGKEVVAKGWGQRHPLAGPGVPGPPTEWTFIYAPRNEEELAVVEMIIEASIGYMTN

SEQ ID NO: 25 - protein, 219 aa, synthetic construct
  REGION 1..219: note = Amino acid sequence of the functional fragment of lusiferase from Mycena citricolor (23-241)
FPFIKKDYETFLKGGPSYAPQNVRGYIIVLVLALFRQEQLGLEIYDRMPEKRRWLANLPQREGPRPKTTSHIIQRQLSQHTDPAFGAAYLKDTVIPRVQARHAANTHIARSTFEFHAAAIFLNADVPLPEGLPASETVRRTKGEIAHMHDYHDFTLHLALAAADGKEVVGKGWGQRHPLAGPGVPGPPNEWTFVYAPRNEEEMGVVEQIVEAAIGYMSN

SEQ ID NO: 26 - protein, 221 aa, synthetic construct
  REGION 1..221: note = Amino acid sequence of the functional fragment of lusiferase from Neonothopanus gardneri (32-252)
FPFIRRDYQTFLEVGPSYAPQNFRGYIIVCVLSLFRQEQKGLEIYDRLPEKRRWLSDLPFRDGPRPSITSHIIQRQRTQLVDPDFATQELIGKVIPRVQARHTDKTFLSTSKFEFHAKAIFLLPSIPINDPLNVPSHDTVRRTKREIAHMHDYHDCTLHIALAAQDGKEVLKKGWGQRHPLAGPGVPGPPTEWTFLYAPRNEEEVRVVEMIIEAAIGYMTN

SEQ ID NO: 27 - protein, 221 aa, synthetic construct
  REGION 1..221: note = Amino acid sequence of the functional fragment of lusiferase from Omphalotus olearius (25-245)
FPFIRKDYQTFLEVGPSYAPQNLQGYIIVCVLSLFRQEQKDVAIYDRLPEKRRWLGDLPFREGPRPSITSHIIQRQRTQLADAEFATKELIGKIIPRVQARHTNTTFLSTSKFEFHAQAIFLLPSIPINDPQNIPSHDTVRRTKREIAHMHDYHDCTLHLALAAQDGKEVLEKGWGQRHPLAGPGVPGPPTEWTFLYAPRSEEEVRVVEMIVEASVVYMTN

SEQ ID NO: 28 - protein, 221 aa, synthetic construct
  REGION 1..221: note = Amino acid sequence of the functional fragment of lusiferase from Panellus stipticus (21-241)
FPFVRKDYHTFLEGGPSYAPQNLQGYIIVLVLSLFRGEETGLEIYDRLPEKRRWLEELPVREGPRPKTTSHIIQRQLNQHVDPDFGMNSLKGSVIRRLQSRHQDITQLALSKFEFHAEAIFLRPDIAINDPKHVPSHDTVRRTKREIAHMHDYHDYTCHLALAAQDGKQVIAKGWGQRHPLAGPGMPGPPTEWTFLYAPRNEAEVQVLETIIEASIGYMSN

SEQ ID NO: 29 - protein, 221 aa, synthetic construct; identical to SEQ ID NO: 19
  REGION 1..221: note = Consensus sequence
  SITE 3: note = MISC_FEATURE - Xaa is Phe, Ile or Tyr.
  SITE 4: note = MISC_FEATURE - Xaa is Ile or Val.
  SITE 5: note = MISC_FEATURE - Xaa is Arg or Lys.
  SITE 6: note = MISC_FEATURE - Xaa is Arg or Lys.
  SITE 9: note = MISC_FEATURE - Xaa is Gln, Glu or His.
  SITE 13: note = misc_feature - Xaa can be any naturally occurring amino acid
  SITE 14: note = MISC_FEATURE - Xaa is Gly or Val.
  SITE 23: note = misc_feature - Xaa can be any naturally occurring amino acid
  SITE 24: note = MISC_FEATURE - Xaa is Arg, Lys or Gln.
  SITE 27: note = MISC_FEATURE - Xaa is Ile Phe or Leu.
  SITE 30: note = MISC_FEATURE - Xaa is Leu or Cys.
  SITE 33: note = MISC_FEATURE - Xaa is Ser or Ala.
  SITE 37: note = MISC_FEATURE - Xaa is Gly or Gln.
  SITE 39: note = MISC_FEATURE - Xaa is Glu or Gln.
  SITE 40: note = MISC_FEATURE - Xaa is Lys, Leu or Thr.
  SITE 41: note = MISC_FEATURE - Xaa is Gly or Asp.
  SITE 42: note = MISC_FEATURE - Xaa is Leu or Val.
  SITE 43: note = MISC_FEATURE - Xaa is Ala or Glu.
  SITE 46: note = MISC_FEATURE - Xaa is Glu or Asp.
  SITE 47: note = MISC_FEATURE - Xaa is Pro or Arg.
  SITE 48: note = MISC_FEATURE - Xaa is Leu or Met.
  SITE 53: note = MISC_FEATURE - Xaa is Thr or Arg.
  SITE 56: note = misc_feature - Xaa can be any naturally occurring amino acid
  SITE 57: note = MISC_FEATURE - Xaa is Glu, Asp or Asn.
  SITE 60: note = misc_feature - Xaa can be any naturally occurring amino acid
  SITE 62: note = misc_feature - Xaa can be any naturally occurring amino acid
  SITE 64: note = MISC_FEATURE - Xaa is Asp, Thr or Pro.
  SITE 67: note = misc_feature - Xaa can be any naturally occurring amino acid
  SITE 68: note = MISC_FEATURE - Xaa is Thr or Ile.
  SITE 77: note = MISC_FEATURE - Xaa is Leu or Arg.
  SITE 78: note = misc_feature - Xaa can be any naturally occurring amino acid
  REGION 80..81: note = misc_feature - Xaa can be any naturally occurring amino acid
  SITE 83: note = MISC_FEATURE - Xaa is Pro, Gln, Leu, Ser or Ala.
  SITE 84: note = misc_feature - Xaa can be any naturally occurring amino acid
  SITE 86: note = MISC_FEATURE - Xaa is Val, Ala or Gly.
  REGION 87..88: note = misc_feature - Xaa can be any naturally occurring amino acid
  SITE 89: note = MISC_FEATURE - Xaa is Ala, Glu, Tyr or Ser.
  SITE 91: note = MISC_FEATURE - Xaa is Lys, or Ile.
  SITE 92: note = misc_feature - Xaa can be any naturally occurring amino acid
  SITE 93: note = MISC_FEATURE - Xaa is Thr, Lys or Ser.
  SITE 94: note = MISC_FEATURE - Xaa is Val or Ile.
  SITE 96: note = MISC_FEATURE - Xaa is Pro or Arg.
  SITE 98: note = MISC_FEATURE - Xaa is Val or Leu.
  SITE 100: note = MISC_FEATURE - Xaa is Ala or Ser.
  SITE 103: note = MISC_FEATURE - Xaa is Thr, Ala or Gln.
  SITE 104: note = MISC_FEATURE - Xaa is Asp, Ala or Asn.
  SITE 105: note = misc_feature - Xaa can be any naturally occurring amino acid
  SITE 107: note = MISC_FEATURE - Xaa is His, Phe or Gln.
  SITE 108: note = MISC_FEATURE - Xaa is Leu or Ile.
  SITE 109: note = MISC_FEATURE - Xaa is Ala or Ser.
  SITE 110: note = MISC_FEATURE - Xaa is Leu, Thr or Arg.
  SITE 112: note = MISC_FEATURE - Xaa is Lys or Thr.
  SITE 118: note = MISC_FEATURE - Xaa is Glu, Lys, Ala or Gln.
  SITE 122: note = MISC_FEATURE - Xaa is Val or Leu.
  SITE 123: note = MISC_FEATURE - Xaa is Arg, Leu, or Asn.
  SITE 124: note = MISC_FEATURE - Xaa is Pro or Ala.
  SITE 125: note = MISC_FEATURE - Xaa is Glu, Ser or Asp.
  SITE 126: note = MISC_FEATURE - Xaa is Ile or Val.
  SITE 127: note = MISC_FEATURE - Xaa is Ala or Pro.
  SITE 128: note = MISC_FEATURE - Xaa is Ile or Leu.
  SITE 129: note = misc_feature - Xaa can be any naturally occurring amino acid or none.
  SITE 130: note = MISC_FEATURE - Xaa is Asp or none.
  SITE 132: note = MISC_FEATURE - Xaa is Lys, Leu, Glu, or Gln.
  SITE 133: note = MISC_FEATURE - Xaa is His, Asn or Gly.
  SITE 134: note = misc_feature - Xaa can be any naturally occurring amino acid
  SITE 136: note = MISC_FEATURE - Xaa is Ser or Ala.
  SITE 137: note = MISC_FEATURE - Xaa is His or Ser.
  SITE 138: note = MISC_FEATURE - Xaa is Asp or Glu.
  SITE 145: note = MISC_FEATURE - Xaa is Arg or Gly.
  SITE 156: note = misc_feature - Xaa can be any naturally occurring amino acid
  SITE 158: note = MISC_FEATURE - Xaa is Leu or Cys.
  SITE 160: note = MISC_FEATURE - Xaa is Leu or Ile.
  SITE 165: note = MISC_FEATURE - Xaa is Gln or Ala.
  SITE 167: note = MISC_FEATURE - Xaa is Ala or Gly.
  SITE 169: note = MISC_FEATURE - Xaa is Gln or Glu.
  REGION 171..172: note = misc_feature - Xaa can be any naturally occurring amino acid
  SITE 186: note = misc_feature - Xaa can be any naturally occurring amino acid
  SITE 191: note = misc_feature - Xaa can be any naturally occurring amino acid
  SITE 196: note = misc_feature - Xaa can be any naturally occurring amino acid
  SITE 201: note = MISC_FEATURE - Xaa is Thr, Asn or Ser.
  SITE 203: note = MISC_FEATURE - Xaa is Glu or Ala.
  REGION 205..206: note = misc_feature - Xaa can be any naturally occurring amino acid
  SITE 208: note = MISC_FEATURE - Xaa is Val or Leu.
  SITE 210: note = MISC_FEATURE - Xaa is Thr, Met or Gln.
  SITE 212: note = misc_feature - Xaa can be any naturally occurring amino acid
  SITE 215: note = MISC_FEATURE - Xaa is Ser or Ala.
  SITE 216: note = MISC_FEATURE - Xaa is Ile or Val.
  SITE 217: note = MISC_FEATURE - Xaa is Ala, Gly or Val.
  SITE 220: note = MISC_FEATURE - Xaa is Thr or Ser.